(19) United States
(12) Patent Application Publication
(10) Pub. No.: US 2023/0064374 A1
(43) Pub. Date: Mar. 2, 2023

(54) APPARATUS AND METHODS TO PROVIDE A SCALABLE AND FLEXIBLE HIGH CHANNEL DENSITY NEURAL INTERFACE

(71) Applicants: Blackrock Microsystems, LLC, Salt Lake City, UT (US); The Trustees of Columbia University in the City of New York, New York, NY (US)

(72) Inventors: Moritz Michael LEBER, Salt Lake City, UT (US); Florian SOLZBACHER, Salt Lake City, UT (US); Ken SHEPARD, Ossining, NY (US)

(21) Appl. No.: 17/898,882

(22) Filed: Aug. 30, 2022

Related U.S. Application Data
(60) Provisional application No. 63/238,485, filed on Aug. 30, 2021.

(51) Int. Cl. A61N 1/36; A61N 1/372

(52) U.S. Cl. CPC A61N 1/36185; A61N 1/37247

(57) ABSTRACT

An apparatus includes a flexible, electrically conducting layer disposed between a first flexible electrically insulating layer and a second flexible electrically insulating layer. At least one of the first electrically insulating layer, the electrically conducting layer, or the second electrically insulating layer includes multiple recesses defined therein, the recesses collectively having a predefined pattern. The apparatus also includes protrusions in the form of penetrating beam structures capable of accessing deep tissue structures, and at least one electrical access site electrically coupled to the electrically conducting layer, forming an electrode site. The apparatus also includes microelectronic circuitry/coils and components to serve as an implantable, flexible, conformally adjustable, interface for stimulating and/or recording electrical activity in biological tissue.

### CROSS-REFERENCE TO RELATED APPLICATIONS

This application claims priority to and the benefit of U.S. Provisional App. No. 63/238,485, filed on Aug. 30, 2021 and titled “APPARATUS AND METHODS TO PROVIDE A SCALABLE AND FLEXIBLE HIGH CHANNEL DENSITY NEURAL INTERFACE,” which is incorporated herein by reference in its entirety for all purposes.

### BACKGROUND

Advances in medical science have made it possible to sense and/or modulate the activity of a neural system (e.g., brain) of an organism. These advances have allowed scientists and researchers to interact and observe a neural system in more detail, including through the use of machines designed to either stimulate the human neural system, or to observe or record its functions.

These advances, however, have been limited in their implementation and restricted to a narrow scope of clinical application. The limitation is largely due to the restrictive and/or potentially invasive properties of the instruments used to access, stimulate and/or record signals from a neural system with a desired degree of impact. Thus, there exists a need for technological improvements in the instrumentation and the implementation of the instrumentation used to stimulate and/or record signals from a neural system, in the form of advanced neural interfaces. Such advanced neural interfaces may then be used to provide advanced treatments for illnesses associated with a nervous system, as well as to study and/or improve a performance of a healthy nervous system.

### SUMMARY

This disclosure relates to the fabrication and use of neural lace apparatuses configured to interface with a neural system. Embodiments include an apparatus that includes a flexible electrically conducting layer disposed between a flexible first electrically insulating layer and a flexible second electrically insulating layer. At least one of the first electrically insulating layer, the electrically conducting layer, or the second electrically insulating layer include a plurality of recesses defined therein, the plurality of recesses having a predefined pattern. A portion of the electrically conducting layer is exposed via an electrical access site in at least one of the first electrically insulating layer or the second electrically insulating layer.

Embodiments disclosed include a method comprising providing an apparatus in a first, folded configuration. The apparatus includes a flexible substrate having an electrically conductive layer disposed between a flexible first electrically insulating layer and a flexible second electrically insulating layer. At least one of the first electrically insulating layer, the electrically conducting layer, or the second electrically insulating layer includes a plurality of recesses defined therein, the plurality of recesses having a predefined pattern. The method further comprises introducing the apparatus into a biological environment of a patient, thereby causing the apparatus to transition to a second, unfolded configuration, such that the apparatus substantially conforms to a non-planar surface within the biological environment of the patient.

### BRIEF DESCRIPTION OF THE DRAWINGS

FIG. 1 is a schematic block diagram representation of a neural lace apparatus, according to an embodiment.

FIG. 2 illustrates an application of a neural lace apparatus as an interface with a human brain, according to an embodiment.

FIG. 3 illustrates a perspective view of a neural lace apparatus, according to an embodiment, when placed on a surface of a neural tissue, according to an implementation.

FIGS. 4A-4E show example lace or mesh patterns that can be implemented using recesses in a neural lace apparatus, according to an embodiment.

FIGS. 5A-5E are schematic illustrations of five different types of protrusions that can be defined on a neural lace apparatus, according to various embodiments.

FIG. 6A is a perspective view of a neural lace apparatus that can be used to interface with a neural tissue, according to an embodiment.

FIG. 6B is a magnified view of a portion of the neural lace apparatus shown in FIG. 6A.

FIG. 7A is a perspective view of a neural lace apparatus to be placed on a surface of a neural tissue, according to an embodiment.

FIG. 7B is a magnified view of a portion of the neural lace apparatus shown in FIG. 7A, including a plurality of electrical access sites that can serve as electrode contact areas.

FIG. 8 is a schematic representation of a neural interface system including a neural lace apparatus, according to an example implementation.

FIG. 9 is a flowchart showing a method of coating and introducing a neural lace apparatus in a biological environment, according to some embodiments.

FIG. 10 is a schematic representation of a fabrication process for producing a lace handling demonstrator that mimics a neural lace apparatus, according to some embodiments.

FIG. 11 shows multiple magnification views of a layout of neural lace apparatuses prior to singulation, according to some embodiments.

FIG. 12 is a schematic representation of a first fabrication process for producing a neural lace apparatus with protrusions, according to some embodiments.

FIG. 13 is a schematic representation of a second fabrication process for producing a neural lace apparatus with protrusions, according to some embodiments.

FIG. 14 shows two example configurations of protrusions of a neural lace apparatus, according to some embodiments.

FIGS. 15A-15D show example arrangements of protrusions within a given neural lace apparatus, according to some embodiments.

FIG. 16 is a photographic image of a neural lace apparatus, according to some embodiments.

### DETAILED DESCRIPTION

Systems and methods of the disclosure relate to fabrication and use of a “neural lace” apparatus to effectively interface with a neural system and provide methods and/or instrumentation to observe and record signals derived from neural activity and/or to deliver signals that can stimulate and/or cause the generation of neural activity in an organism. As used herein, the phrase “neural lace” refers to an apparatus including a mesh or other open-structured, flexible, electrically conductive structure, with optional functionality embedded therein/thereon, and configured for use as a brain-computer interface (BCI). Some embodiments of the apparatuses and methods described herein can be used for in-vivo implementations, i.e., to interface with a neural system of a live organism. Some embodiments of the apparatuses and methods described herein can be used to interface with a neural system of a live organism that is awake and performing a behavior.

In some embodiments, the disclosed neural lace apparatus and methods can be used for interfacing with a central nervous system of a human or animal patient undergoing a treatment for a disorder associated with the patient's nervous system. In some embodiments, the disclosed neural lace apparatus, and the associated methods of use of the neural lace apparatus can be used to provide an interface to the neural system of an individual (human or animal) to study, observe, and/or activate (e.g., stimulate or modulate) physiological activity in the neural system.

In recent years, advances in medical science have allowed access to modulate and sense the activity of the neural systems of humans and animals. These advances have allowed scientists and researchers to interact and observe the various neural systems in more detail. Some approaches to interacting with and observing various neural systems include the use of processing machines configured to observe or record neural activity, and in some cases even stimulate neural activity. The various approaches have varying scope and accompanying limitations in terms of degree of access, specificity, precision, and/or accuracy of recording signals and/or delivering signals that stimulate neural activity.

A neural system of an organism includes neural tissue that has specific electrochemical properties such that electro-chemical transfer of information between and/or among cells in the neural tissue (e.g., neurons) can be observed, recorded and/or manipulated by external sources. For example, transfer of information between neurons, also referred to as neural activity, can include electrical signatures or signals that can be captured and/or recorded by a suitable apparatus included in a neural interface system. Similarly, externally generated chemical, optical and/or electrical signals (also referred to as stimuli) designed using electronic processing systems can be delivered via a neural interface to the neural tissue to stimulate or cause the generation and/or modulation of neural activity in the neural tissue. For example, neural activity can be manipulated using chemical, optical, or electrical stimulus provided by one or more external sources such as neurochemical modulators, light emitters, stimulating electrodes and/or the like. Such neural interface systems can use the chemical, optical and/or electrical signals to exchange information with a neural system providing a Neural-Machine-Interface (in some instances referred to as “Brain Computer Interface” (BCI) or “Human Machine Interface (HMI)) for communication and/or control.

One example method of electrically stimulating neurons in a neural tissue involves inducing a change in an electrical differential across the cell membrane of one or more neurons using an applied or external electrical field. In some instances, the change in the electrical differential in turn can initiate a chain reaction of electrical activity in the stimulated one or more neurons referred to as an action potential. This initial chain reaction can cause subsequent similar depolarization in neurons nearby as the electrical activity gets electrochemically transmitted between neurons and/or neuronal populations that are connected to the stimulated one or more neurons. This transmitted neural activity can activate a large-scale chain reaction of similar action potentials throughout a region in the neural tissue and in some instances can recruit an entire nervous system into being active. Such a chain reaction, depending on the size of activation, can cause an electrical field in a localized (or large) area within the neural tissue, also referred to as a local field potential, which can be observed, recorded, and/or registered using suitable electrical sensing and/or recording devices.

The electrical stimulation of neural tissue can be accomplished using a variety of electrical stimulators, including microelectrodes implanted in neural tissue and surface area electrodes placed on a subject's skin in proximity to the neural tissue that is to be stimulated. Inversely, neural activity can be recorded from nervous tissue through implantable microelectrodes and surface electrodes.

A burgeoning field of research into the function and operation of the neural systems has emerged and is centered on stimulating and/or recording neural activity from the human and/or animal nervous system. This research, however, has been limited by the ability of stimulating and recording from a neural system with sufficient precision, accuracy, and/or specificity. Impediments in the study of neural systems also include an inability to stimulate and/or record a wide range of neural activity varying in magnitude, spread in area of neural tissue, and/or type or profile of activity (e.g., spatiotemporal profile of activity). A demand thus exists for improvements in methods and apparatus that increase the precision, accuracy, and/or specificity with which activity in a neuron or a neural tissue may be stimulated and/or observed. Furthermore, a need exists for improvements in methods and apparatus that allow precision, accuracy, and/or specificity of stimulation and/or recording a wide range of neural activity as described previously.

Improvements in the technology involved in stimulating and/or recording activity in neurons have generally been greatly limited by the properties of the instruments used to stimulate and/or record activity in a neural system.

One example device that can be used to generate instrumentation for stimulating and/or recording neural activity includes embedded ASIC chips used to generate high-density channels that can serve as neural interfaces. Such embedded ASIC chips can be generated by thinning a silicon substrate down to a specified thickness, where silicon transitions from a rigid behavior to a flexible behavior. This change to a flexible behavior allows fabrication of an implantable flexible microelectronic system within or in proximity to a neural tissue.

Implementing neural interfaces that are formed using the current form of embedded ASIC chips can, however, have several considerations. Firstly, the ASIC chips may be spatially limited in surface area and depth. This limits the access for stimulating and/or recording neural activity to a superficial depth in a neural tissue. For example, using the surface area and/or depth limited ASIC chips limits access to the outer most layers of a human or animal brain due to non-penetrating surface electrodes, prohibiting access to deeper layers of neural tissue. Secondly, the neural interfaces may include continuous, non-disrupted material films. The continuous and non-disrupted nature of the film, to be implanted over or in proximity to a neural tissue, can limit the overall surface area of a neural system that can be covered by a single device and thus be accessible for stimulating and/or recording. Additionally, the continuous nature of the film when implanted in or proximal to neural tissue can interrupt, disrupt, or block cellular processes in the neural tissue such as cross flow of cellular fluids like the cerebrospinal fluid or physiological fluids in the extracellular and/or intracellular matrix of the neural tissue. In some instances, such disruptions or blockages can negatively affect tissue health and thus impact suitability and/or acute or chronic usability of the apparatus or device. For example, biological tissue commonly exhibits a foreign body response following implants, regardless of types of implants. Typically, such a foreign body response can include persistent inflammation at the interface of the implant and is to me managed and/or controlled for effective continued use of the implant. Thirdly, the ASIC chips are complex to process and produce. They further require multiple post-processing and packaging steps to form a biocompatible encapsulating layer over the device, protecting the microelectronic device from the biological environment and vice versa, prior to implantation using specified methods.

Embodiments described herein include a neural lace apparatus that is configured to serve as a neural interface or a brain-computer interface (BCI) and may be used to record from and/or stimulate neural tissue. The neural lace apparatus can be configured to address the aforementioned considerations of spatial limitation (e.g., limitations in area, depth, expanse,) including potential limitations in usability or suitability for implantation with regard to tissue health. Embodiments of the neural lace apparatus can be configured to address process complexity limitations mentioned above.

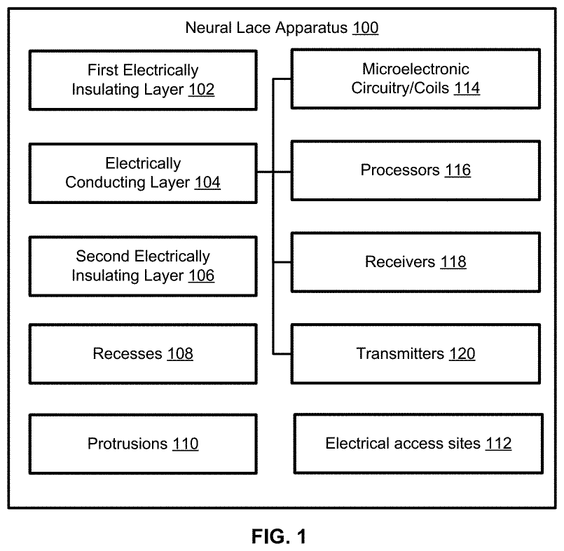

FIG. 1 is a schematic representation of a neural lace apparatus 100, in the form of a block diagram, according to an embodiment. The neural lace apparatus 100 can be configured to be implanted in, over, or proximal to a neural tissue that is to be monitored or recorded from. In some embodiments, the neural lace apparatus 100 can be used to observe and/or record naturally occurring neural activity in the neural tissue. In some embodiments, the neural lace apparatus 100 can be used to stimulate the neural tissue to generate and/or modulate a desired neural activity using predefined electrical, optical, and/or chemical signals. In some embodiments, a neural lace apparatus 100 can be configured to observe, record preexisting neural activity as well as stimulate the neural tissue to generate or modulate a desired neural activity.

In some embodiments, the neural lace apparatus 100 can be configured to be used as an acute implant in a biological tissue, for example in a central nervous system or a peripheral nervous system of a subject. In some embodiments, the apparatus 100 can be configured to be used as a chronic implant configured to function for an elongated period of time.

The neural lace apparatus 100 can be constructed using any suitable design and/or manufacturing process to generate electronic circuitry including silicon-based device manufacturing, semi-conductor chip fabrication, fabrication and/or assembly of flexible electronics, design, and fabrication of integrated circuits (e.g., application specific integrated circuits (ASIC)), and/or the like.

In some embodiments, the neural lace apparatus 100 can be configured to be implanted in a physiological environment within a subject (e.g., human or animal subject) to interface with neural tissue and be coupled to an external electrical/electronic system via any suitable method. For example, in some embodiments, the neural lace apparatus 100 can be configured to be coupled to an external electrical/electronic instrumentation via wired connection. In some embodiments, the neural lace apparatus 100 can be configured to interface with neural tissue and be coupled to external electrical/electronic instrumentation wirelessly. In some embodiments, the neural lace apparatus 100 can be configured to couple to external instrumentation via a combination of wired and/or wireless methods.

In some embodiments, the neural lace apparatus 100 can be configured to cover and/or interface with a desired portion of a neural tissue (e.g., a human or an animal brain). In some embodiments, the neural apparatus 100 can be configured to cover or interface a portion of neural tissue that incudes easily visible or accessible regions as well as regions that are not easily visible and/or not easily accessible, including for example, neural tissue that may be hidden beneath other tissue structure or folds (e.g., neural tissue hidden in sulci or fissures) as described in further detail herein.

The neural lace apparatus 100 can be configured to be implanted in a subject using any suitable procedure of a suitable degree of intensive or invasive interaction with a subject. For example, in some instances a neural lace apparatus 100 can be configured to be implanted using intensive surgical procedure. As another example, in some instances, a neural lace apparatus 100 may be suitable for implantation in a minimally invasive (e.g., outpatient) procedure.

The neural lace apparatus 100 can be configured to maintain a desired degree of functionality upon implantation with a desired degree of biocompatibility, accessibility to neural tissue, precision, and/or accuracy of operation (e.g., recording, stimulating). In some instances, the neural lace apparatus 100 can be configured to have a reduced surface area to reduce a foreign body response that is a physiological response commonly observed in association with implanted devices. For example, the neural lace apparatus 100 can be configured to have an improved biocompatibility such that acute and/or chronic implantation results in a reduced instance of persistent inflammation at a biotic-abiotic interface between biological tissue and the apparatus.

Persistent inflammation and/or foreign body response can be measured using indicators like a presence of biomarkers signaling an immune response. In some instances, soluble factors secreted by cells can shape the foreign body reaction and can be responsible for dysfunction in the neural tissue and/or a blood brain barrier (BBB). In some instances, the dysfunction may be caused by a mixture of released factors (e.g., proinflammatory and cytotoxic cytokines such as IL-1B, IL-6, and TNF-a). One method of reducing the impact of such released factors is to induce less release by reducing area of contact. Another method of reducing the impact of released factors is by allowing for immediate clearance of the released factors from the localized region of neural tissue.

An improved biocompatibility of the neural lace apparatus 100 can be achieved through improved size, shape, structure, materials, surface area, and/or penetration profile. In some embodiments, the neural lace apparatus 100 can be shaped to have a thin flexible structure with reduced surface area in the form of a lace, mesh, or lattice to induce a reduced immune response or foreign body response (e.g., reduced macrophage activation at a biotic-abiotic interface). In some embodiments, the neural lace apparatus 100 can include one or more recesses that allow cross flow of cellular fluids or other biological milieu in the tissue, to improve the clearance of soluble factors released by cells in the tissue (e.g., immune cells, cells in a blood-brain barrier, etc.) that may otherwise cause inflammation when localized.

In some embodiments, the neural lace apparatus 100 can be configured to be flexible to conformally adjust to a tissue structure upon implantation to increase accessibility to regions of a neural tissue (e.g., a brain surface having raised gyri and crevasses in sulci).

As shown in FIG. 1, the neural lace apparatus 100 includes a first electrically insulating layer 102, an electrically conducting layer 104, and a second electrically insulating layer 106. Embodiments of the neural lace apparatus include one or more recesses 108, one or more protrusions 110 (also referred to herein as needles or cantilevered protrusions), and one or more electrical access sites 112, as described in further detail in the following sections. Embodiments of the neural lace apparatus 100 further include microelectronic circuitry/coils 114, one or more processors 116, one or more receivers 118, and one or more transmitters 120, each of which can be configured to be electrically coupled to the electrically conducting layer 104, as shown in the schematic in FIG. 1.

The first electrically insulating layer 102, the electrically conducting layer 104, and the second electrically insulating layer 106 can be configured such that the electrically conducting layer 104 is encapsulated or sandwiched by the first electrically insulating layer 102 and the second electrically insulating layer 106. In some embodiments, the electrically conducting layer 104 enclosed by the first electrically insulating layer 102 and the second electrically insulating layer 106 can collectively form a substrate of the neural lace apparatus 100. In some embodiments, the first and second electrically insulating layers can encapsulate more layers in addition to the electrically conducting layer 104. For example, the neural lace apparatus 100 can include two or more electrically conducting layers 104 and one or more additional electrically insulating layers (not shown in FIG. 1) with each electrically conducting layer separated from an adjoining electrically conducting layer by an electrically insulating layer acting as a dielectric layer (e.g., two or more distinct electrically conducting layers configured to include and/or allow definition of separated or distinct electrical circuitry, not shown in FIG. 1). In some embodiments, one or more of the additional electrically insulating layers can be patterned to serve as traces of insulating material with opening allowing electrical access sites 112 and/or recesses 108, defined to support and/or help define microelectronic circuitry/coils 114, processors 116, receivers 118, transmitters 120, and/or any suitable components included in the neural lace apparatus 100 (e.g., capacitive elements, inductive elements, conductive elements, and/or the like). The multiple electrically conducting layers and the one or more electrically insulating layers can be collectively enclosed by or encapsulated by the first electrically insulating layer 102 and the second electrically insulating layer 106.

The first electrically insulating layer 102 and the second electrically insulating layer 106 can be configured to enclose the one or more electrically conducting layer(s) 104 and the optional, additional one or more electrically insulating layers such that the enclosed layers are protected from an external environment such as a biological or physiological environment in which the neural lace apparatus 100 may be implanted to be used, for example, as a neural interface.

The first electrically insulating layer 102 and/or the second electrically insulating layer 106 can be of any suitable thickness and comprise of or be formed from any suitable material that can serve as a biofluid barrier for an electronic system. For example, in some embodiments, the first electrically insulating layer 102 and/or the second electrically insulating layer 106 can be a thickness of 10 nm-10 μm. In some embodiments, the first electrically insulating layer 102 and/or the second electrically insulating layer 106 can be formed from a material that is specifically selected for suitability to generate flexible electronics for implantation. In some embodiments, the material for forming the first electrically insulating layer 102 and/or the second electrically insulating layer 106 can be selected based on one or more specific criteria including (i) biocompatible molecular composition; (ii) high electrical capacitance (for electrical interfaces); (iii) low thermal conductivity and thermal mass (considered for potential thermal interfaces); (iv) good optical transparency (to provide for optical interfaces); (v) low areal mass density (for minimized inertial load); (vi) low flexural rigidity (to allow conformal integration onto curved surfaces); (vii) defect-free, material perfection over large areas (e.g., over several square centimeters); (viii) thermal and chemical compatibility with polymer substrates (to provide suitability for device fabrication) and (ix) lifetimes of long duration (e.g., multiple years and/or decades) in electrolyte solutions at physiological pH and temperature, under cyclic bending conditions (for robust operation throughout the life of a patient).

The first electrically insulating layer 102 and/or the second electrically insulating layer 106 can be formed from any suitable material including polyimide, silicon carbide, SU-8, a liquid crystal polymer (LCP), Parylene-C, a ceramic, silicon dioxide, or any combination thereof. The first electrically insulating layer 102 and/or the second electrically insulating layer 106 can be fabricated or formed using any suitable process. For example, in some embodiments, the electrically insulating layer 102 and/or the second electrically insulating layer 106 can be formed from an ultrathin layer of thermal silicon oxide which can be transfer bonded.

The first electrically insulating layer 102 and/or the second electrically insulating layer 106 can be formed to meet any suitable shape guidelines. In some embodiments, the first electrically insulating layer 102 and/or the second electrically insulating layer 106 can be patterned to include openings defining electrical access sites 112 and/or recesses 108, as described herein in further detail below. In some embodiments, portions of the first electrically insulating layer 102 and/or the second electrically insulating layer 106 can be formed to include one or more protrusions 110 that can extend in any suitable dimension and provide for depth access as described in further detail herein.

The electrically conducting layer 104 can be encapsulated by the first electrically insulating layer 102 on one side and the second electrically insulating layer 106 on the other side. The electrically conducting layer 104 can be formed to include electrode traces, and/or regions configured to serve as electrodes. The electrically conducting layer 104 can be of any thickness and formed using any suitable material.

In some embodiments, the electrically conducting layer 104 can be formed as a set of electrical traces or regions that are electrically conductive and form, support and/or couple two or more elements or components included in the neural apparatus 100. The electrically conducting layer 104 can be of any suitable thickness and/or width. For example, in some embodiments, the electrically conducting layer 104 can be a thickness of 50 nm-5 μm. The electrically conducting layer 104 can comprise of or be formed from any suitable material including gold, platinum, iridium, iridium oxide, titanium nitride, poly(3,4-ethylenedioxythiophene (PEDOT), carbon nano-tubes (CNT), doped silicon including highly doped silicon (p++ and p−), or a combination thereof.

In some embodiments, the electrically conducting layer 104 can extend into peripheral or interspersed microelectronic circuitry 114 to define an interconnected web of peripheral microelectronics circuitry/coils 114, with interspersed processors 116 (e.g., application specific integrated circuits or ASICs) at certain locations (e.g., nodes) on the interconnected web of microelectronics circuitry/coils 114. In some embodiments, the electrically conducting layer 104 can extend into interspersed protrusions 110 or penetrating beam structures forming electrode arrays which may be used to stimulate and/or record neural activity.

In some embodiments, the electrically conducting layer 104 can be patterned to electrically couple microelectronic elements, ASIC units, memory units, logic units, coil components, optoelectronic components (e.g., light emitting diodes or photo-diodes), and/or any combination thereof. These elements may be used for signal processing and/or data transmission to an external device instrumentation system. In some embodiments, the electrically conducting layer 104 can be patterned to electrically couple other elements that may be coupled to or integrated in the apparatus 100 including processing units, controller units, multiplexers, de-multiplexers, buffers, analog/digital converters, digital/analog converters, or any combination thereof.

As described, previously, in some embodiments, the electrically conducting layer 104 can include multiple electrically conducting layers (i.e., two or more electrically conducting layers) with one or more additional electrically insulating layers separating adjoining electrically conducting layers. The one or more additional electrically insulating layers can be configured to be dielectric layers that isolate the adjoining electrically conducting layer. The two or more distinct electrically conducting layers 104 separated by electrically insulating layers (or dielectric layers) can be configured to include and/or allow definition of separated or distinct electrical circuitry (not shown in FIG. 1).

In some embodiments, the two or more distinct electrically conducting layers 104 can each be patterned to include distinct electrode traces including distinct electrically conducting contact points or electrode access sites 112. The electrode traces, the electrically conducting contact points, and/or the electrode access sites 112 can be used to serve as electrodes in contact with a tissue structure, and/or as contact points to couple to, support, and/or help define microelectronic components (e.g., ASICs, capacitive elements, inductive elements, conductive elements, and/or the like), microelectronic circuitry/coils 114, processors 116, receivers 118 and/or transmitters 120 included in the neural lace apparatus 100. The two or more distinct electrically conducting layers 104, which can be separated by intermediately disposed electrically insulating layers, can be collectively encapsulated by the first electrically insulating layer 102 and the second electrically insulating layer 106 to form a flexible neural lace apparatus 100 that can conformally adjust to a surface of a tissue structure, upon implantation, providing increased access to the underlying tissue.

The neural lace apparatus 100 can include one or more recesses 108 defined in the first electrically insulating layer 102, the second electrically insulating layer 106, and/or the electrically conducting layer 104. In some embodiments, the one or more recesses 108 can further be defined in any number of additional electrically conducting and/or electrically insulating layers encapsulated by the first electrically insulating layer 102 and the second electrically insulating layer 106. In some embodiments, the neural lace apparatus 100 can include a plurality of recesses 108. Each recess from the plurality of recesses 108 can be of any shape, size, and or depth (e.g., as observed in a plan view, a cross-sectional view, and/or the like). For example, a recess 108 can be of a square shape, a rectangular shape, a circular shape, an oval shape, or a polygonal shape, as observed in a plan view. The plurality of recesses 108 can be configured to generate an increased flexibility and/or pliability of the neural lace apparatus 100.

In some embodiments, at least one recess from the plurality of recesses 108 can be partially defined in each of the first electrically insulating layer 102, the electrically conducting layer 104, and the second electrically insulating layer 106, such that the at least one recess extends through each of the first electrically insulating layer 102, the electrically conducting layer 104, and the second electrically insulating layer 106 (and any other intervening additional layers enclosed by the first electrically insulating layer 102 and the second electrically insulating layer 106), generating a through via, aperture, or hole. Said in another way, in some embodiments, a set of recesses 108 can be defined to form through holes that allow cross flow of substances (e.g., extracellular milieu in biological tissue upon implantation of the apparatus 100). In some embodiments, a neural lace apparatus 100 including recesses 110 defined according to such a predefined pattern can be rendered to adopt a structural form that can have a suitable flexibility and/or ability to conform to irregular three-dimensional surface (e.g., gyri and/or sulci of a portion of a human and/or animal brain).

In some embodiments, the plurality of recesses 108 can be configured to improve biocompatibility of the apparatus 100 by reducing a surface area of the apparatus 100 and/or to reduce an area of contact between the apparatus 100 and a biological tissue to form a biotic—abiotic interface. In some embodiments, the plurality of recesses 108 can be configured to extend through each of the first electrically insulating layer 102, the electrically conducting layer 104, and the second electrically insulating layer 106 (and any other additional layers enclosed by the first electrically insulating layer 102 and the second electrically insulating layer 106) to generate a plurality of vias or passages through a thickness of the neural lace apparatus 100 to allow cross flow of biological fluids. In some instances, such a plurality of recesses 108 generating vias or passages through a thickness of the apparatus 100 can permit improved biocompatibility of the apparatus 100 is implanted by supporting cross flow of biological fluids mediating improved clearance of potentially cytotoxic factors released from cells (e.g., immune cells, and/or injured cells from iatrogenic impact of implantation, etc.).

In some embodiments, a plurality of recesses 108 can be arranged according to a predefined pattern. A predefined pattern can be any suitable pattern including that of a mesh or a lace geometry. The neural lace apparatus 100 can include a predefined lace or mesh pattern of recesses 110 such that the apparatus 100 can effectively conform to the contours of the brain surface. The lace or mesh pattern of the apparatus 100 can thereby increase a potential surface area of a biological tissue that may be covered with the apparatus 100 upon implantation.

In some instances, a predefined pattern of recesses 108 can include repeating recesses or recess units of one or more shapes forming a lattice structure generating a mesh or lace geometry. In some embodiments, the pattern can include recesses 108 having repeating units of one or more shapes. In some embodiments, the pattern of recesses 108 can include repeating recess units of any suitable shape (e.g., square, rectangle, circle, oval, polygon and/or the like). In some embodiments, the pattern of recesses 108 can include a tessellated pattern of recesses 108 (also referred to as recess units) covering a surface area of a substrate (e.g., a substrate formed by a planar portion of the apparatus 100 including the first electrically insulating layer 102, the electrically conducting layer 104, and the second electrically insulating layer 106) of a neural apparatus 100.

In some embodiments, the neural lace apparatus 100 can include recesses 108 according to a single predefined pattern. The pattern may be selected based on a desired pattern of stimulating and/or recording neural activity in a tissue upon implantation. In some instances, the pattern may be selected based on a physiology of a target tissue (e.g., a structure of the tissue, a foreign body response expected upon implantation, a degree of access to the tissue that is desired, a target implementation of the neural apparatus (e.g., a treatment plan, a diagnostic plan, and/or the like), etc.) In some embodiments, the set of recesses 108 can form a lattice structure that includes nodes.

In some embodiments, the neural lace apparatus 100 can include recesses 108 according to a plurality of predefined patterns. That is, a pattern of recesses 108 can include a first pattern of recesses (e.g., defined by repeating recess units of a first specified shape and/or size) located at a first region in the apparatus 100 and a second pattern of recesses (e.g., defined by repeating recess units of a second specified shape and/or size) located at a second region in the apparatus 100. In some embodiments, the various patterns and/or location of each pattern on an apparatus 100 can be selected based on a desired usage or implementation (e.g., a target neural tissue that each pattern and/or location of the apparatus is desired to be placed proximal to and/or stimulate activity/record activity from). As another example, a first set of recesses 108 according to a first pattern can extend through the thickness of a substrate (e.g., first electrically insulating layer 102, the electrically conducting layer 104, and the second electrically insulating layer 106 and any additional layer therebetween) of a neural apparatus 100 providing vias, while a second set of recesses 108 according to a second pattern can extend partially through the thickness of a substrate (e.g., just the first electrically insulating layer 102, or just the second electrically insulating layer 106).

The neural lace apparatus 100 can include one or more protrusions 110 defined to protrude from a plane defined by a substrate or base portion of the apparatus (e.g., a planar portion of the first electrically insulating layer 102, the electrically conducting layer 104, and the second electrically insulating layer 106). In some embodiments, the protrusions 110 can be formed to include a portion of the electrically conducting layer 104 and a portion of at least one of the first electrically insulating layer 102 or the second electrically insulating layer 106. For example, in some embodiments, a protrusion 110 can be shaped as a penetrating beam structure with the beam structure including a portion of the electrically conducting layer 104 protruding or extending from a planar region along a direction away from the planar region.

In some embodiments, the protrusion 110 can be formed to extend from a surface of a planar portion of the neural lace apparatus 100 with the protruding portion of the electrically conducting layer 104 being encapsulated by at least one of the first electrically insulating layer 102 or the second electrically insulating layer 106. In some embodiments, the neural lace apparatus 100 can include a plurality of protrusions 110 the plurality of protrusions 110 including beam structures formed by protruding portions of the electrically conducting layer 102. The plurality of protrusions 110 can be configured to extend from the planar portion of the substrate on one side of the planar portion (e.g., a side that may be facing a neural tissue during implantation). The plurality of protrusions 110 can be encapsulated by either the first electrically insulating layer 102 (e.g., if the protrusions extend from the side proximal to the first electrically insulating layer 102) or encapsulated by the second electrically insulating layer 106 (e.g., if the protrusions extend from the side proximal to the second electrically insulating layer 106). The plurality of protrusions 110 can extend along any suitable direction that defines an angle of between 5° and 175° with respect to a plane defined by an adjacent planar portion of the apparatus.

In some embodiments, the protrusions 110 may be bent or folded out of plane mechanically, through intrinsic stress, epitaxial stress, thermal stress, or shape memory alloys. In some embodiments, the protrusions 110 may be pushed out of plane using a solid structure such as styli, beams, pins, needles, arrays, or any combination thereof. The beam structures may penetrate the brain tissue.

In some embodiments, the protrusions 110 can be coated with a rigid material to provide sufficient mechanical stability for insertion into biological tissue. This material may consist of any biocompatible (silicon carbide), biodegradable, or dissolvable material (e.g., polyethylene glycol). The material providing mechanical stability for insertion may be coated with an additional biocompatible layer. A protrusion 110 may be coated with an additional insulating layer in addition to the encapsulating material layer used in a fan-out process. In some instances, additional layers may be deposited using micromachining techniques, including but not limited to chemical vapor deposition, physical vapor deposition, dip coating, spin coating, or electrodeposition, such as electro-polymerization and electroplating.

A protrusion 110 may exhibit or include one or more electrode sites, that are electrically connected to the electrically conducting layer, and exposed to an external environment (e.g., a biological environment upon implantation). As an example, the protrusions 110 may be connected to embedded electronic components (e.g., processors 116, receivers 118, transmitters 120, etc.) of the neural lace apparatus 100.

In some embodiments, the protrusions 110 can be configured to be penetrating beam structures that include electrical access sites 112 at any number of variable positions along the lengths of each of the protrusions 110. In some embodiments, the plurality of protrusions 110 can form electrode arrays with the electrical access sites 112 accessing varying depths when the apparatus 100 is implanted such that the protrusions 110 are made to impinge into neural tissue (e.g., brain tissue). In some embodiments, the length of the protrusions 110 can be determined based on a desired degree of access to target structures within a depth of a neural tissue. Some such embodiments can be used to greatly increase the depth at which a tissue structure (e.g., neural tissue within a brain) may be stimulated and/or the activity may be recorded.

The protrusions 110 can be of any suitable shape or size having any suitable length and/or width. The protrusions 110 can each have any suitable longitudinal shape profile (i.e., a shape profile along a longitudinal axis of a protrusion) including cylindrical, conical, cuboidal, polygonal pyramidal, and/or the like. The protrusions 110 can each have any suitable cross-sectional shape profile (i.e., a shape profile along a cross section of a protrusion) including a circle, oval, square, polygon, and/or the like.

In some embodiments, the protrusions 110 can have any shape profile that may be suitable for a particular implementation. In some embodiments, one or more of the protrusions 110 can have one or more non-varying and/or uniform shape profiles. In some embodiments, one or more of the protrusions 110 can have one or more varying shape profiles that vary along one or more axes or directions defined on the protrusion 110 (e.g., along a longitudinal axis, a transverse axis, a radial axis, and/or a circumferential axis, etc.). In some embodiments, one or more of the protrusions 110 can have one or more longitudinal portions, also referred to herein as segments. Each longitudinal portion or segment can be defined to have a distinct non-varying or varying shape profile, as desired. In some embodiments, one or more protrusions 110 can have a distal tip portion configured to have a distinct shape. For example, protrusions 110 can have a distal tip that is shaped to be rounded or narrow width to reduce potential impact on a tissue during impalement of the protrusion 110 as the apparatus 100 is implanted in the tissue.

In some embodiments, a neural lace apparatus 100 can have a plurality of protrusions 110 having the same shape profile. In some embodiments, a neural lace apparatus 100 can include a plurality of protrusions 110 of which a first set of protrusions have a first shape profile, and a second set of protrusions have a second shape profile and so on.

In some embodiments, the apparatus 100 can include a plurality of protrusions 110 that includes a first set of protrusions 110 having a first length and a second set of protrusions 110 having a second length. For example, an apparatus 100 can include a subset of protrusions 110 that are specifically located (e.g., on a specified portion of the apparatus 100) and configured (e.g., to have a specified length and/or shape profile, with a specified set of electrical access sites 112 located at various positions, etc.) to target a deeper tissue structure, compared to the other protrusions 110, for stimulating and/or recording activity.

As described herein, the apparatus 100 includes one or more electrical access sites 112 that can be configured to provide direct or indirect access to the electrically conducting layer 104 and/or other electrical components included in the apparatus 100. The electrical access sites 112 can be of any shape and/or size and be formed using any suitable procedure. The electrical access sites 112 can be located on any suitable portion of the apparatus 100.

In some embodiments, the one or more electrical access sites 112 can be defined in the first electrically insulating layer 102, and/or the second electrically insulating layer 106, to expose an underlying portion of the electrically conducting layer 104. In some embodiments, one or more electrical access sites 112 can be defined without any openings in the first electrically insulating layer 102 or the second electrically insulating layer 106. That is, the one or more electrical access sites 112 can be defined to be encapsulated by the first electrically insulating layer 102 or the second electrically insulating layer 106.

In some embodiments, the electrical access sites 112 can be configured to provide direct or indirect electrical access between the electrically conducting layer 104 of the apparatus 100 and a tissue structure or a component of the apparatus 100 (e.g., a microelectronic circuitry/coil 114, a processor 116, a receiver 118, a transmitter 120, etc.). In some embodiments, the electrical access sites 112 can be provided via openings defined on at least one of the first electrically insulating layer 102 or the second electrically insulating layer 106 such that an underlying portion of the electrically conducting layer 104 can be exposed via the opening defining an electrical access site 112. Such an electrical access site 112 can be configured to allow at least a portion of the exposed electrically conducting layer 104 to serve as an electrode terminal or an electrode site that can be in electrical contact with a portion of the neural tissue, or an electrical contact point to provide electrical coupling of the electrically conducting layer 104 with an electrical/electronic circuit and/or component.

In some embodiments, the electrical access site 112 can be configured to provide electrical access to tissue (e.g., neural tissue) upon implantation, for stimulating and/or recording electrical activity in the tissue. In some instances, the first electrically insulating layer 102 or the second electrically insulating layer 106 may be patterned to expose portions of the electrically conducting layer 104 to an external environment, forming an electrical access site 112.

In some embodiments, the electrical access sites 112 can be configured to provide conductive electrical coupling with the tissue. The electrical access sites 112 can be configured to provide conductively coupled interfaces using any suitable process. For example, in some embodiments, the electrical access sites 112 can be formed using methods that exploit patterns of highly doped silicon nanomembranes chemically bonded to thin, thermally grown layers of SiO2 as leakage-free, chronically stable, conductively coupled interfaces. In some embodiments, the electrode sites 112 and/or regions associated with electrode sites 112 can have one or more coatings encapsulating the surface of the electrode sites 112 and/or regions associated with electrode sites 112 such that they do not come in direct interface with a biological environment upon implantation. For example, in some embodiments, the highly doped silicon in electrode sites 112 and/or regions associated with the electrode sites 112 can be configured to include a coating with a suitable materials such that they do not directly interface with biological tissue. For example, in some embodiments, the coatings can include gold films interfacing with p⁺⁺-Si. The materials that can be used for such coatings can include gold, platinum, iridium, iridium oxide, titanium nitride, poly(3,4-ethylenedioxythiophene (PEDOT), carbon nano-tubes (CNT), or a combination thereof. Additional details can be found in “Conductively coupled flexible silicon electronic systems for chronic neural electrophysiology” by Li et. al, published in the Proceedings of the National Academy of Sciences October 2018, 115 (41) E9542-E9549, the entire contents of which are incorporated herein by reference in their entireties for all purposes. The electrical access sites 112 and/or the apparatus 100 incorporating the electrical access sites 112 providing conductive coupling can be fabricated using any suitable process available to a person of ordinary skill in the art of fabrication of electronic, and/or microelectronic devices. For example, any suitable fabrication process to generate flexible electrical interfaces for any purpose including implantation as a bioelectrical interfaces.

In some embodiments, electrical access site 112 can be configured to provide indirect (or non-contact based) electrical access to tissue (e.g., neural tissue) upon implantation, for stimulating and/or recording electrical activity in the tissue. In some instances, an electrical access site 112 can be capacitively coupled to biological tissue in an external environment through an insulating layer of material. The electrical access sites 112 can be configured to provide capacitively coupled interfaces using any suitable process. For example, the electrical access sites 112 can be configured to transfer charge between the biological ionic conducting environment and the solid-state electronic medium of the electrically conducting layer 104 of the neural lace apparatus 100. In some embodiments, the electrode sites 112 may consist of an additional electrically conducting layer. For example, in some embodiments, the electrical access sites 112 can be formed using ultrathin, thermally-grown layer of silicon dioxide that can form a substantial portion of the apparatus 100 (e.g., without any openings defined in the first electrically insulating layer 102 or the second electrically insulating layer 106), and serve both as a dielectric to facilitate direct capacitive coupling to a set of semiconducting channels in arrays of silicon nanomembrane (Si NM) transistors included in the apparatus 100 and as a robust, biocompatible barrier layer to prevent penetration of bio-fluids. As an example, the apparatus 100 can include Si n-channel metal-oxide semiconductor (NMOS) transistors defined on a silicon on insulator (SOI) wafer. A set of dielectric or electrically insulating layers and a set of metal or electrically conducting layers can be defined to provide interconnects and electrical access sites to access the sensing electrodes that are configured to interface with tissue. The sensing electrodes can include multiplexed capacitive sensors placed at specified locations on the apparatus 100 and each sensor can include underlying Si NM transistors (e.g., two Si NM transistors) one of which connects to a metal pad that is coupled to an electrically conducting layer 102 from its gate electrode. A layer of thermally grown silicon dioxide (e.g., 900 nm, SiO2) can cover the entire outer surface of the apparatus 100. In some embodiments, the layer of thermally grown silicon dioxide can have any suitable thickness from 5 nm to 50 μm or any number therebetween. In some embodiments, bonding a layer of polyimide over all the components or electronics included in the apparatus 100 can yield a thin, flexible neural lace apparatus 100 upon removal of the silicon wafer. In some embodiments, portions of the electrical access sites 112 can be formed using a layer of thermal oxide layer. In some embodiments, a layer of silicon carbide layer (SiC) can be used to form portions of electrical access sites 112. Additional details can be found in “Capacitively coupled arrays of multiplexed flexible silicon transistors for long-term cardiac electrophysiology” by Fang et. al, published at Nature Biomedical Engineering 1, 0038 (2017), the entire contents of which are incorporated herein by reference in their entireties for all purposes.

As described previously, the electrical access sites 112 can be located at any portion of the neural lace apparatus 100 that includes the first electrically insulating layer 102 or the second insulating layer 106. In some embodiments the electrical access sites 112 located on protrusions 110 (e.g., at various positions along a length of a protrusion) can serve as electrode sites 112 and may be used to interface with the tissue upon implantation to stimulate and/or record neural (or other electrical) activity in the tissue (at varying depths of penetration). The locations of the one or more electrical access sites 112 serving as electrode sites on the protrusions 110 may be manipulated to target stimulation at different depths of a neural tissue in which the apparatus 100 may be implanted. The beam cross section of the protrusions 110 and the electrode placement of the electrical access sites 112 may produce different stimulation and/or recording parameters. In some embodiments, the various electrode sites may be used for current and charge steering applications.

In some embodiments, the electrical access sites 112 can include electrode terminals or electrode sites that are located at interspersed locations on the substrate or base portion (the first electrically insulating layer 102, the electrically conducting layer 104, and the second electrically insulating layer 106) of the neural lace apparatus 100. For example, electrical access sites can be located on nodes formed by the lattice structure defined by the set of recesses 108. In some embodiments, some such electrical access site 112 can be configured to provide electrical access to other components included in the apparatus 100. In some embodiments, using an Integrated Fan-Out (InFO) technology, the electrical access sites 112 may be directly connected to the one or more microelectronic components, including microelectronic circuitry/coils 114, processors 116, receivers 118, transmitters 120, etc., used for stimulation and/or recording of electrical activity associated with a biological tissue.

The microelectronic circuitry/coils 114 in the neural lace apparatus 100 can include patterned electrical traces, electrodes, and/or dielectric layers. In some embodiments, the material layers (e.g., the electrically insulating layers 102 and/or 106, and/or the electrically conducting layer 104) may be patterned to form microelectronic components, such as coils, or capacitive elements. Such patterning can be performed, for example, using one or more of: photolithography, wet etching (e.g., wet chemical etching), or dry etching (e.g., plasma etching).

The apparatus 100 can include a set of processors 116. The processors 116 can be directly or indirectly electrically coupled to other components of the apparatus 100 and/or external devices via the electrically conducting layer 104 of the apparatus 100. The processors 116 included in the apparatus can be any suitable processor that can be configured to receive electrical signals, perform desired computations on the signals, and return processed electrical signals. As an example, the processors 116 can include ASICs or ASIC chips generated by thinning the silicon substrate down to a thickness, where silicon transitions from a rigid to flexible behavior, allowing the fabrication of implantable flexible microelectronic systems.

The apparatus 100 can include a set of receivers 118 configured to receive signals (e.g., data, power, etc.) from external devices either via wired connections or via wireless data/power transmission methods. The apparatus 100 can include a set of transmitters 120 configured to transmit signals (e.g., data, power, etc.) to external devices either via wired connections or via wireless data/power transmission methods.

In some embodiments, the microelectronic circuitry/coils 114 may form an interconnected web of peripheral microelectronics circuits and/or coils, with interspersed processors 116 or application specific integrated circuits at certain locations on the interconnected web of microelectronic circuitry/coils 114, with interspersed protrusions 110 forming electrode arrays which may be used to stimulate and/or record neural activity. In some embodiments, the microelectronic circuitry/coils 114 may include a coil pattern used for transdermal and transcranial data transmission, telemetry, power supply, or any combination thereof. The transmitted signal may be modulated in any suitable manner (e.g., pulse-width modulation, pulse-amplitude modulation, pulse-position modulation) as desired.

In some embodiments, the neural lace apparatus 100 can include any number of suitable components or elements not shown in FIG. 1, such as microelectronic devices, ASICs, memory, logic, and/or coil components, optoelectronic components, (e.g., light emitting diodes or photo-diodes), or any combination thereof. The various elements can be connected together via the electrically conducting layer 104. These elements may be used for signal processing, and/or data transmission to an external compute device or instrumentation system. In some embodiments, the elements may further include other processing units, controller units, multiplexers, de-multiplexers, buffers, analog/digital converters, digital/analog converters, or any combination thereof. In some embodiments, for example, a coil pattern may be used for transdermal and transcranial data transmission, telemetry, power supply, or any combination thereof. The transmitted signal may be modulated, (e.g., pulse-width modulation, pulse-amplitude modulation, pulse-position modulation). In some embodiments, the transmitters 120 and/or receivers 118 of the neural lace apparatus 100 may include one or more elements for data and information transmission using ultrasound or optical technology (e.g., ultrasonic emitter, an ultrasonic receiver, etc.).

In some embodiments, as described herein, one or more elements of the described microelectronic circuitry/coils 114 (e.g., ASIC processors, or coil components) may be integrated into the neural lace apparatus 100, as desired. The desired components may be integrated at any location on the neural lace apparatus 100, for example at nodes defined by the recesses 108 in a lace pattern and/or on a peripheral area of the neural lace apparatus 100.

In some implementations, a neural lace manufacturing process can include the following: the first electrically insulating layer 102 and/or the second electrically insulating layer 106 can be formed by depositing an ultrathin layer of silicon dioxide (SiO₂) onto a silicon substrate, the SiO₂ obtained using any suitable process, such as physical vapor deposition, thermal oxidation, or chemical vapor deposition. For example, thermal oxidation of a silicon wafer can be performed, e.g., at a temperature of about 1,100° C. The oxidation process can be followed by transfer printing of electrode traces, for example using a pattern of gold, to form the electrically conducting layer 102. Next, the metallized oxidized silicon substrate can be transferred by bonding a top surface of the metallized oxidized silicon substrate onto a thin polymer film (e.g., polyimide, 25 micrometers (μm) thick) disposed on a glass substrate, where the glass substrate serves as a temporary, rigid support to facilitate manual manipulation. The remaining silicon can subsequently be removed using a single dry etching step or a combination of multiple different dry etching steps. Peeling the bonded metallized oxidized silicon from the temporary glass substrate support can result in a flexible electronic neural lace apparatus encapsulated across its entire front or top surface with a layer of thermal SiO₂ as a defect-free barrier to biofluids, with stability for potential chronic implantation. Similar growth of SiO₂ and transfer of a polyimide film, as discussed above, can also be performed to produce a layer of SiO₂ on the back or bottom surface of the flexible apparatus, for example to prevent biofluid penetration from the back side of the apparatus. The foregoing process can be scaled to considerably larger silicon wafers (e.g., the largest currently available silicon wafers, at 450-mm diameter), thereby facilitating the manufacture of a neural lace apparatus 100 that can provide substantial coverage of area across a target neural tissue (e.g., a surface of a brain). Such a process for forming an implantable apparatus with a biofluid barrier can be compatible with cost effective manufacturing techniques, since in some instances, the cost of semiconductor manufacturing per unit area can decrease with increasing wafer sizes.

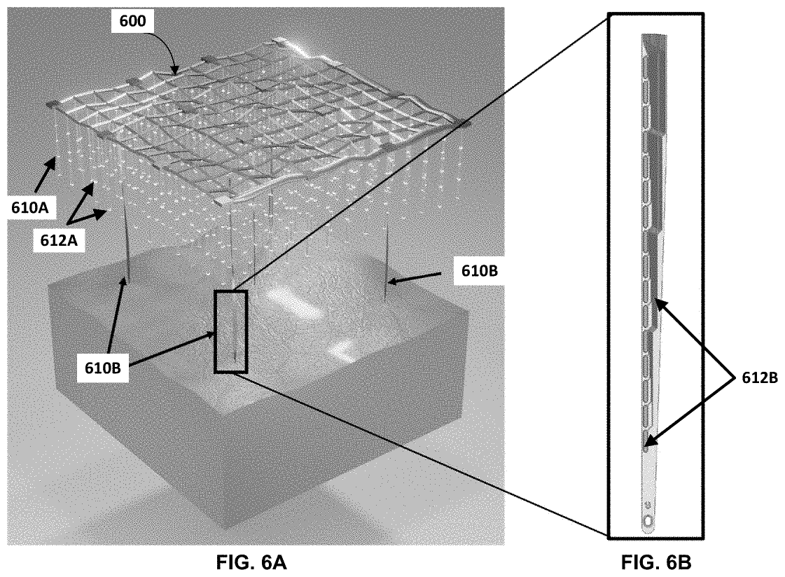

In some embodiments, an apparatus 100 can include one or more separately fabricated additional components exhibiting one or more electrodes that may be attached to the neural lace apparatus 100. For example, in some implementations, an apparatus 100 can include one set of protrusions 110 having a first length that are fabricated with the apparatus 100, and a second set of protrusions 110 having a second length that extends beyond the first length (e.g., the protrusions 610B shown in FIGS. 6A and 6B). Such extended protrusions 110 can be generated by attaching a set of electrode arrays to a subset of protrusions 110 having the first length as part of a post-fabrication step.

Any number of additional components, which may include electrode arrays, or optoelectronic components used for optical stimulation of nervous tissue, etc., may be attached to the apparatus 100 using any suitable method including lamination, gluing, chemical, or diffusive technologies, and/or the like. The additional components may exhibit one or more electrically conducting layers, which can form an electrical interface to one or more electrically conducting layers of the neural lace apparatus 100. The additional components may be single electrodes or electrode arrays of variable dimensions that can be used as a range extender, e.g., to target biological tissue at various depths relative to the surface of the apparatus 100. In some embodiments, additional features (e.g., structures with lumen for fluid flow, structures to actuate, mediate, and/or control fluid flow, etc.) configured for drug delivery purposes may be attached for fluid or substance delivery purposes. In some embodiments, additional features may be included to be used as optical guides or for optical stimulation purposes.

In some instances, fan-out wafer level technology can be used to allow packaging of single microelectronic dies or ASICs directly on a wafer level during a fabrication of the apparatus 100. Fan-out packaging technology may be used for wafer level encapsulation of the neural lace apparatus 100 to protect any enclosed circuitry, microelectronic components, and or ASICs from the biological environment upon implantation. Fan-out packaging technology can also significantly reduce process complexity in the manufacturing of a high channel count and large-scale neural interface, with little to no post processing being required.

In some instances, the neural lace apparatus 100 can be encapsulated using an encapsulating material layer that may be patterned through micromachining processes, including but not limited to dry etching, wet etching, dicing, to form a mesh or lace type geometry. Material in areas which do not contain any microelectronic components or circuitry may be removed, as desired, to reduce a total surface area of biological tissue being in contact with the apparatus 100 as a foreign material. Such a reduced surface areas via the lace or mesh pattern of the apparatus 100 can reduce the foreign body response and allow for much larger area coverage on a tissue (e.g., the brain), allowing free circulation of biological fluids (e.g., cerebrospinal fluid).

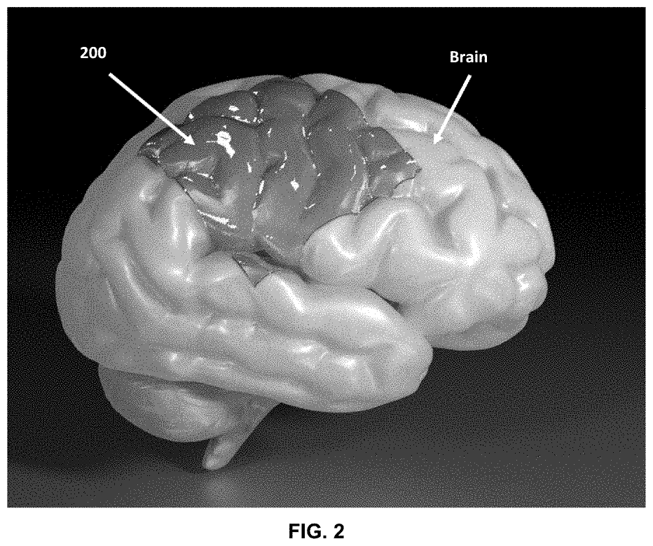

FIG. 2 is a schematic of neural lace apparatus 200, that can be substantially similar in structure and/or function, to the neural lace apparatus 100. As shown in FIG. 2, the neural lace apparatus 200 can be configured to conformally adjust (conform to gyri and/or sulci) to a surface of an underlying brain tissue upon implantation over a surface of the brain (e.g., over a dura or a pia mater of the brain). The neural lace apparatus 200 can be implanted on the surface of a human brain, for example, above or below the dura, pia, and/or the arachnoid matter, such that the neural lace apparatus 200 can conform to an underlying neural tissue. As shown in FIG. 2, when implanted over or proximal to a parietal lobe of a human brain, the lace or mesh geometry of the recesses in the neural lace apparatus 200 can permit the neural lace apparatus 200 to conform to a large spatial (e.g., areal) expanse of neural tissue including neural tissue defined by protruding portions or gyri (e.g., post central gyms, supramarginal gyms, etc.) and sunken portions of neural tissue or sulci (e.g., central sulcus, postcentral sulcus, Sylvian fissure, etc.) which may be otherwise out of access of a neural interface apparatus provide electrical access

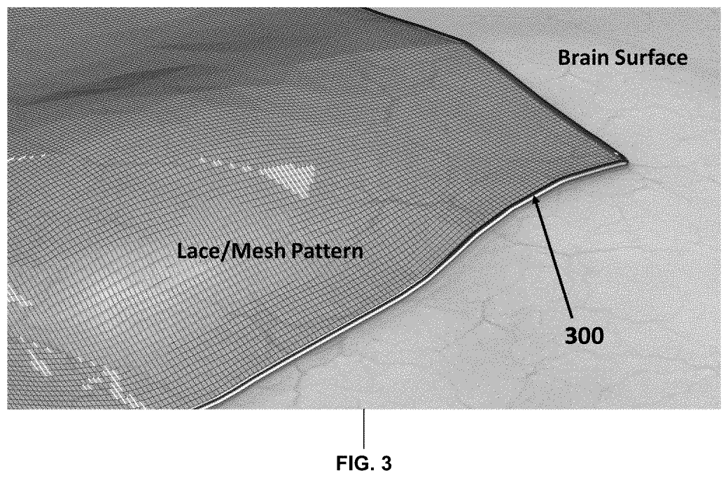

FIG. 3 is a schematic representation of a magnified view of a neural apparatus 300 implanted on or over a brain tissue, according to an embodiment. The neural lace apparatus 300 can be substantially similar in structure and/or function to the apparatus 100 and/or the apparatus 200. As described with reference to the apparatus 100 in FIG. 1, in some embodiments, a plurality of recesses included in the neural lace apparatus 300 can be arranged according to a predefined pattern. A predefined pattern can be any suitable pattern including that of a mesh or a lace geometry. The neural lace apparatus 300 is shown to include a predefined lace or mesh pattern of recesses (which can be similar to the recess 110 of the apparatus 100) such that the neural lace apparatus 300 can be flexible or pliable and effectively conform to the contours of the brain surface. The neural apparatus 300 is shown to be formed to include a plurality of rectangular recesses in a lace or mesh pattern forming a lattice structure. The lace or mesh pattern can allow the apparatus 300 to be flexible or pliable and capable of conformally adjusting to a surface of the brain tissue that may include convoluted structures. The lace or mesh pattern of the apparatus 300 can therefore significantly increase a potential surface area of the brain tissue that may be covered and/or accessed by the apparatus 300 upon implantation.

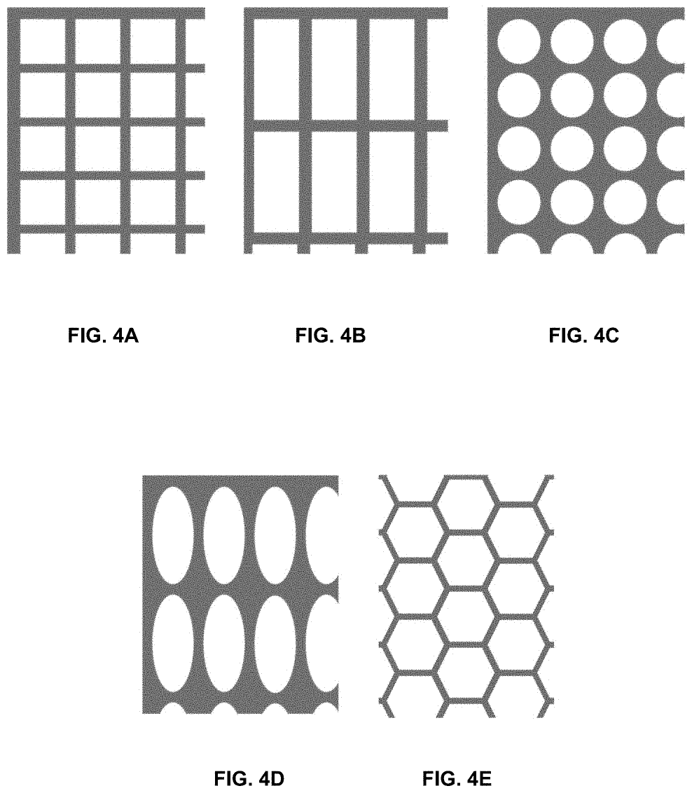

FIGS. 4A-4E show illustrations of five example patterns that can be used to form recesses in a neural apparatus, according to some embodiments. The example patterns are shown to have repeating recess units of varying shapes as described herein. For example, FIG. 4A shows a predefined pattern of recesses including repeating square recess units. FIG. 4B shows a pattern of repeating rectangular recess units, FIG. 4C shows a pattern of repeating circular recess units, FIG. 4D shows a pattern of repeating oval recess units, and FIG. 4E shows a pattern of repeating hexagonal recess units.

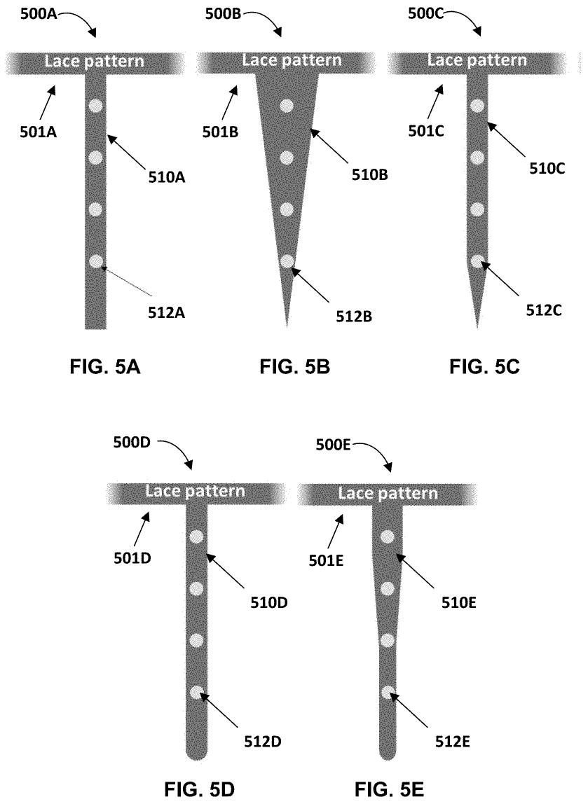

FIGS. 5A-5E, and FIGS. 6A-6B illustrate some examples of protrusions with various shape profiles that can be used in neural lace apparatuses, according to some embodiments. The illustrations in FIGS. 5A-5B show a side view of five example protrusions 510A, 510B, 510C, 510D, and 510E, included in apparatuses 500A, 500B, 500C, 500D, and/or 500E respectively. The protrusions 510A, 510B, 510C, 510D, and/or 510E, can be substantially similar in structure and/or function to the protrusions 110 described above with reference to the apparatus 100 shown in FIG. 1. The protrusions 510A, 510B, 510C, 510D, and 510E, can be included in neural apparatuses 500A, 500B, 500C, 500D, and 500E, respectively. The apparatuses 500A, 500B, 500C, 500D, and/or 500E can be substantially similar in structure and/or function to the apparatuses 100, 200, and/or 300 described herein.

As shown in FIGS. 5A-5E, the protrusions 510A has a uniform shape profile with a substantially uniform width or thickness (e.g., fixed radius is cylindrical or a fixed width if rectangular) along the entire length of the protrusion 510A from a base portion 501A of the apparatus 500A to a second end distal to the base portion 501A. Whereas the protrusion 510B has a varying shape profile that is tapered long the longitudinal axis to have reducing cross-sectional area along a length and/or along the longitudinal axis of the protrusion 510B. The protrusion 510B has a wider profile (e.g., larger cross-sectional area) at a first end of the protrusion 510B proximal to a base portion 501B (e.g., a planar portion of a substrate of a neural lace apparatus 500B) and a narrow profile (e.g., smaller cross-sectional area) at a second end of the protrusion 510B distal to the base portion 501B of the apparatus 500B with a gradual change in cross-sectional area in the intermediate portion between the first end and the second end. Protrusion 510C is shown to a uniform shape profile (e.g., fixed cross-sectional area along a length of the segment) in a first segment proximal to the base portion 501C that extends for a first length of the protrusion 510C and a varying (e.g., tapered) shape profile (e.g., varying cross-sectional area along a length of the segment) in a second segment distal from the base portion 501C that extends for a second length of the protrusion 510C. The protrusion 510D has a uniform shape profile along the length with a distal end or tip portion of the protrusion 510D that is shown to be rounded. In other embodiments, the tip portion could be any suitable shape as desired. The protrusion 510 E has three segments along the length. A first segment proximal to the base portion 501E having a first length, a third segment distal to the base portion 501E having a third length, and a second segment between the first and third segments and having a second length. The first segment has a uniform profile with a first cross-sectional area. The second segment adjacent to the first segment has a varying shape profile along its length with a varying range of second cross-sectional areas of the second segment. The second range of cross-sectional areas is configured to vary between the first cross-sectional area of the first segment and a third cross-sectional area of the third segment. The third segment is shown to have a uniform profile with a third cross-sectional area. The protrusion 510E also includes a distal end or tip portion that is shown to be rounded.

As described previously with reference to the apparatus 100 in FIG. 1, the protrusions in a neural lace apparatus can include one or more electrical access and the electrical access sites can be interspersed at various locations on the one or more protrusions. The protrusions 510A, 510B, 510C, 510D, and 510E each have a set of electrical access sites, for example electrical access sites 512A, 512B, 512C, 512D, and 512E indicated in FIGS. 5A-5E. The illustrations of the example apparatuses in FIGS. 5A-5E show the locations of the interspersed electrical access sites. The electrical access sites can be located at any suitable depth or length of a protrusion to facilitate recording and/or stimulating electrical activity in tissue at varying depths upon implantation of the apparatus.

FIG. 6A is a schematic illustration of an example neural lace apparatus 600 that includes a plurality of protrusions, according to an embodiment. The apparatus 600 can be substantially similar in structure and/or function to the apparatuses 100, 200, 300, and/or 500A-500E. The apparatus 600 includes a plurality of protrusions that can be substantially similar in structure and/or function to the protrusions 110 and/or 510A-510E described herein. The plurality of protrusions in the apparatus 600 includes a first set of protrusions 610A having a first length, and a second set of protrusions 610B having a second length greater than the first length. The second set of protrusions 610B can be configured to extend the range of access of the implanted apparatus 600 into deeper tissue regions. The protrusions 610A and 610B can include at least two sets of electrical access sites 612A and 612B. The protrusions 610A are of the first length, having a first set of electrical access sites 612A interspersed along their length, and configured to access tissue at a first set of locations lying within a first depth upon implantation of the apparatus 600. The protrusions 610B are of the second length, greater than the first length and extending past the first length of the protrusions 610A. The protrusions 610B have a second set of electrical access sites 612B that are interspersed along a portion of their second length that is extending past the first length of the protrusions 610A, and can be configured to access tissue at a second set of locations that may lie at or within a second depth upon implantation of the apparatus 600. FIG. 6B shows a magnified view of a protrusion 610B configured to extend the depth or range of penetration of the apparatus 600. The magnified view of the protrusion 610B shows an example set of sixteen distinct electrical access sites 612B positioned along the length of the protrusion 610B. The set of sixteen electrical sites can be used as a 16-channel penetrating electrode array to probe, stimulate activity, and/or record activity from deeper tissue structure of a neural tissue (e.g., deep brain structures in a human brain). In some instances, the protrusions 610B can be formed or fabricated separately and attached to an apparatus 600 (e.g., attached to the protrusions 610A) using any suitable method as described herein.

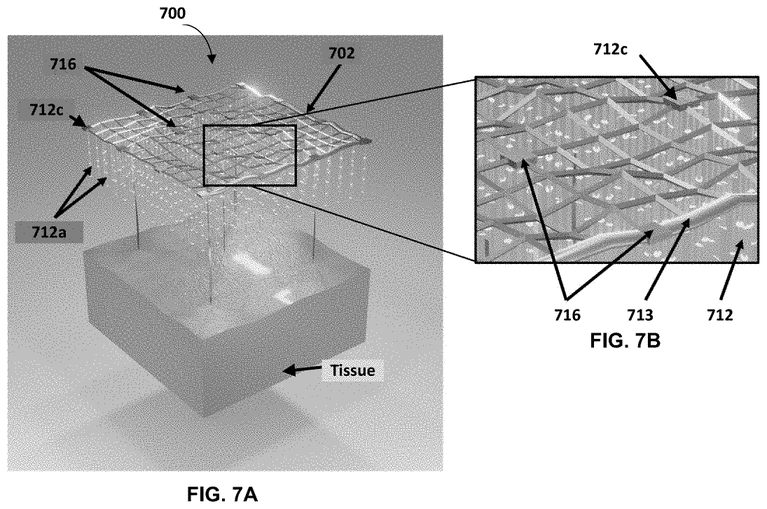

FIGS. 7A and 7B are schematic illustrations of a neural lace apparatus 700 with some example types of electrical access sites 712C located on a node or base portion of the apparatus 700. The electrical access sites 712C can be configured to provide electrical access between an electrically conducting layer and a processor 716 (e.g., an ASIC). FIG. 7B shows a magnified view of the apparatus 700 indicating the processors 716, and the circuitry integrated into a peripheral area 713 of the apparatus 700.

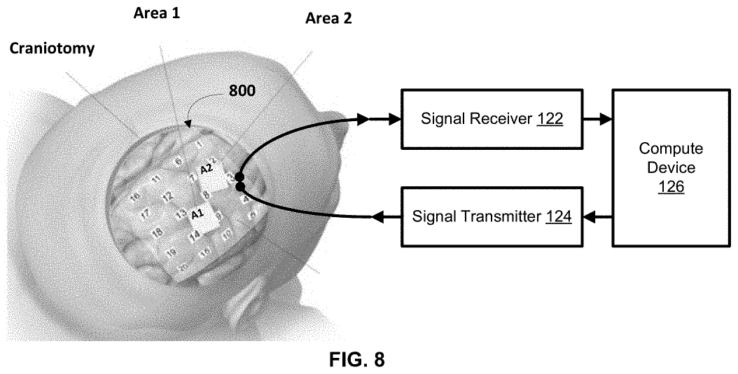

FIG. 8 is a schematic illustration of an interface system incorporating a neural lace apparatus 800 that has been implanted in the brain of a subject via a craniotomy, according to an implementation. The apparatus 800 can be substantially similar in structure and/or function to the apparatuses 100, 200, 300, 500A-500E, 600, and/or 700 described herein. The apparatus 800 is shown to be implanted over the subject's brain to cover areal and area 2 and to conformally adjust to provide increased access and coverage of the underlying brain structures. The interface system of FIG. 8 includes a signal receiver 122, a compute device 126, and a signal transmitter 124 in operational coupling with the apparatus 800. The signal receiver 122 can be coupled to the apparatus via wire or wireless connection and be configured to receive signals (e.g., data and/or power) from the apparatus 800, and transmit the signals to the compute device 126. The signal transmitter 124 can be coupled to the apparatus 800 via wire or wireless connection and be configured to receive signals from the compute device 126 and transmit apparatus 800, and transmit signals (e.g., data/power) to the apparatus 800. The compute device 126 can be any suitable compute device capable of storing data and processing data according to instructions. In some implementations, the interface of FIG. 8 can be set up such that data and/or power communications between the compute device 126 and the apparatus via the signal receiver 122 and/or the signal transmitter 124 can be done in a near real-time basis as the subject is involved in a behavior or brain state (e.g., undergoing neural activity that is being recorded using the apparatus 800 and/or is subjected to stimulation of neural activity by the apparatus 800). For example, the interface can be used during surgical interventions, or during active behavior by the subject (e.g., as a brain machine interface to complement or augment sensory, motor, and/or cognitive aspects of a behavior of a user), to provide a virtual reality experience to a user, etc.

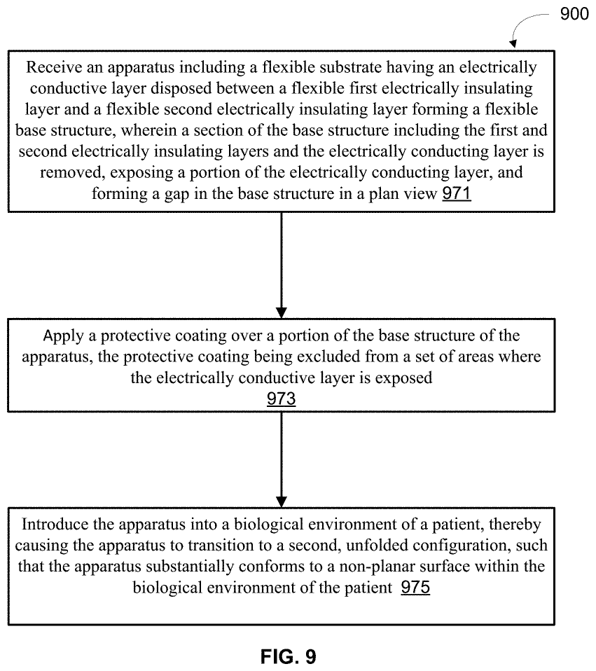

FIG. 9 is a flowchart of a method 900 of preparing and introducing a neural lace apparatus in a biological environment of a subject, according to an implementation. The method 900 can be used to prepare and/or introduce any of the apparatuses described herein including apparatuses 100, 200, 300, 500A-500E, 600, 700, and/or 800.

The method 900 incudes, at 971, receiving an apparatus in a first folded configuration, the apparatus including a flexible substrate having an electrically conductive layer disposed between a flexible first electrically insulating layer and a flexible second electrically insulating layer forming a flexible base structure, at least one of the first electrically insulating layer, the electrically conducting layer, or the second electrically insulating layer including a plurality of recesses defined therein, the plurality of recesses having a predefined pattern. In some instances, the predefined pattern of recesses can be configured to form a set of vias or through holes in the flexible substrate in a plan view.

In some instances, the method can optionally include setting one or more protrusions of the apparatus that are patterned on the base structure such that each beam structure from the one or more protrusions included in the apparatus at a desired angle between 0° and 180°. In some instances, the protrusions may be in a folded configuration (e.g., in an umbrella shape) and may be transitioned to an unfolded configuration to set them at the desired angle. In some implementations, the protrusions may be pushed out of plane using a solid structure such as styli, beams, pins, needles, arrays, or any combination thereof. The protrusions may be suitably configured to penetrate into a desired depth within a target tissue (e.g., brain tissue). The protrusions may include one or more electrical access sites serving as electrode sites that are electrically connected to the electrically conducting layer of the apparatus. As an example, a protrusion may be electrically connected to an embedded ASIC included in the neural lace apparatus.

At 973, the method includes optionally applying a protective coating over a portion of the apparatus, the protective coating being excluded from a set of areas where the electrically conductive layer is exposed. In some instances, the protective coating may be applied over the surfaces of the apparatus except for electrical access sites that may serve as electrode areas configured to come in contact with biological tissue to stimulate and/or record activity from the tissue.

At 975, the method includes introducing the apparatus into a biological environment of a patient, thereby causing the apparatus to transition to a second, unfolded configuration, such that the apparatus substantially conforms to a non-planar surface within the biological environment of the patient. In some instances, the non-planar surface can be a surface of the brain of the patient.

In some instances, the method can include introducing the apparatus into the biological environment of the patient via a craniotomy. In some instances, the introducing the apparatus into the biological environment of the patient can be via a minimally invasive burr hole.

The apparatus can adjust conformally to the brain surface, thereby drastically increasing the potential surface area of the brain that may be covered with the apparatus. Penetrating protrusions or beam structures can form electrode arrays that greatly increase the depth at which the brain may be stimulated and/or the brain activity may be recorded.

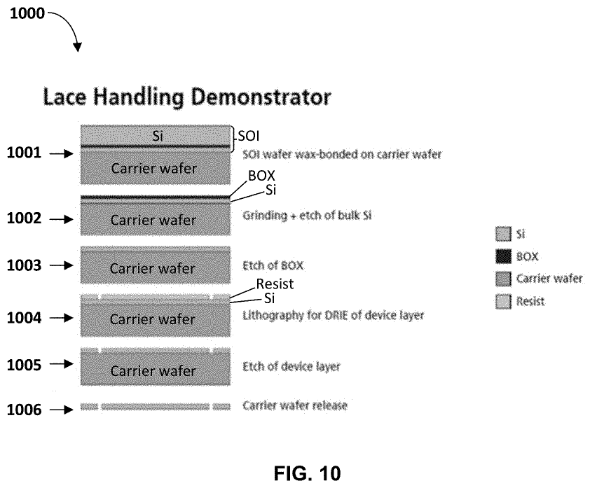

FIG. 10 is a schematic representation of a fabrication process 1000 for producing a lace handling demonstrator, according to some embodiments. The process 1000 can be used as part of a manufacturing process for producing any neural lace apparatus described herein (e.g., neural lace apparatus 100, 200, 300, 600, 700, and/or 800). The lace handling demonstrator can be configured to mimic or simulate a neural lace apparatus described herein (e.g., neural lace apparatus 100, 200, 300, 600, 700, and/or 800). For example, the lace handling demonstrator can mimic the delicate—yet robust for handling—properties of a neural lace apparatus. The lace handling demonstrator 1000 can serve as a mechanical structure for accommodating one or more additional components of a neural lace apparatus. For example, in some embodiments, the lace handling demonstrator can be one of two (or more) back bones or structural elements of a neural lace apparatus. Fabrication of the lace handling demonstrator can be used to develop/refine a process flow for producing neural lace apparatuses, and/or to test the anticipated mechanical properties of a neural lace apparatus. In some embodiments, the lace handling demonstrator 1000 can be used for biocompatibility testing of a neural lace apparatus. In some embodiments, the lace handling demonstrator can lack active electronics, for example, it may not include any ASICs, transistors, or other active elements/components.

As shown in FIG. 10, the process 1000 includes bonding, at 1001, a silicon-on-insulator (SOI) layer to a carrier wafer. as shown in FIG. 10, the SOI layer can include a single crystal silicon layer deposited on or fused to a dielectric (e.g., silicon dioxide (SiO₂)) layer that, in turn, overlies (e.g., bonded to a thermally grown, chemically deposited, vapor deposited, etc.) a silicon substrate (e.g., bulk silicon), collectively forming a buried oxide (BOX) layer. The carrier wafer can be selected and/or configured to provide mechanical support for the structures and/or elements that are to be bonded to it. The carrier wafer can be made of any suitable material (e.g., glass, quartz, silicon, ceramic, another SOI wafer, and/or the like). The bonding can be carried out using any suitable method. For example, in some implementations, the SOI wafer can be wax bonded on to the carrier wafer.

The process 1000 also includes, at 1002, grinding and etching of the bulk silicon to expose the BOX layer. At 1003, the process 1000 includes etching (e.g., wet etching and/or dry etching) the BOX layer. At 1004, the process 1000 includes patterning the single crystal silicon layer (also referred to herein as the “device layer”), for example by applying a layer of photoresist (“resist”) and using lithography and a predefined mask to expose regions of a surface of the single crystal silicon layer, in preparation for deep reactive ion etching (DRIE) of the device layer. In some instances, lithography can be carried out using any suitable approach (e.g., photolithography, electron beam lithography, etc.). At 1005, the process 1000 includes etching the exposed regions of the device layer using DRIE. At 1006, the process 1000 includes removing the carrier wafer to release the device layer. Removal of the carrier wafer can be performed, for example, by dissolving the wax bond. Alternatively, or in addition, removal of the carrier layer can be performed by grinding, dry etching (e.g., plasma etching), wet chemical etching, etc.

A process similar to process 1000, once validated/refined for a particular application, can be used to generate neural lace apparatuses described herein. The manufacture of a lace handling demonstrator and/or a neural lace apparatus, as described herein, can be performed using wafer-scale manufacturing, such that multiple lace devices (i.e., lace handling demonstrators and/or neural lace apparatuses) can be produced in a single run and/or using a single carrier. Each device can be released from the carrier wafer in a final step, e.g., as shown at step 1006 of FIG. 10. In some implementations, each lace device is spaced from each other lace device from a plurality (e.g., an array) of lace devices of a given wafer/stack. In other words, and by way of example, once steps 1001 through 1005 of FIG. 10 have been completed, each device may not be physically connected to any other device within a given layout or on a given carrier wafer, thereby allowing for natural singulation/separation of the devices at the release step. In other implementations, singulation/separation of the devices can be accomplished using a dicing step (e.g., with a dicing saw) or any other method of mechanical division. In some implementations, an outer edge or region of a wafer can remain unetched throughout the fabrication process (e.g., to facilitate handling and/or to improve yield).

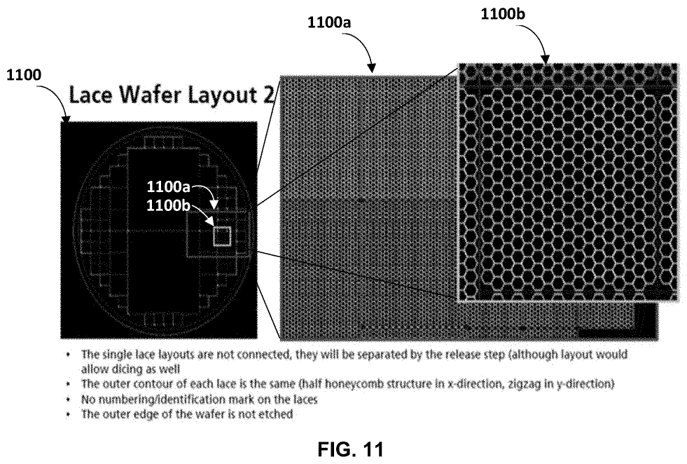

FIG. 11 shows multiple magnification views of a layout of multiple neural lace apparatuses on a wafer, as fabricated and prior to singulation, according to some embodiments. As can be seen in FIG. 11 (see magnified views in 1100a and 1100b), an outer contour of each neural lace apparatus has a common repeating motif of a half honeycomb in the X-axis and a zig-zag shape in the Y-axis. Although shown in FIG. 11 to have an overall substantially square shape, each neural lace apparatus can instead have any other device shape (e.g., polygonal, hexagonal, rectangular, round, oval, oblong, symmetric or asymmetric, etc.). Moreover, for a given plurality of neural lace apparatuses on a wafer during manufacture, a geometry thereof can be uniform across the wafer, or can vary in one or more of: outer contour shape, periodicity of the associated pattern, size of openings/“cells,” or length of connections between openings. The honeycomb structure of each neural lace apparatus can be a regular cubic honeycomb or a quasiregular honeycomb. In other implementations, one or more of the neural lace apparatuses may have an altogether different pattern (i.e., not a honeycomb) with differently shaped cells (e.g., square, round, rectangular, etc.), different packing of cells, tiling density of the cells, etc.

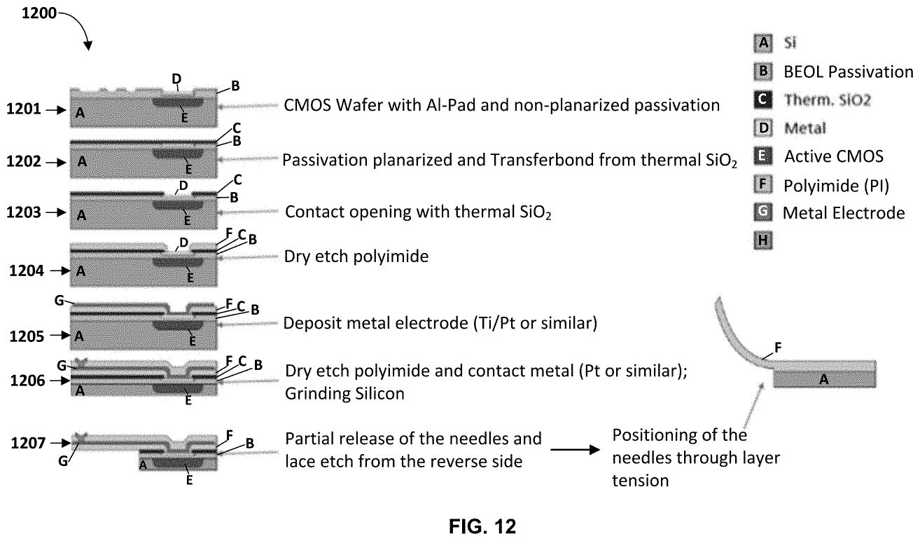

As described herein, in some embodiments, a neural lace apparatus can include one or more protrusions (e.g., protrusions 510A, 510B, 510C, 510D, and 510E, included in apparatuses 500A, 500B, 500C, 500D, and/or 500E respectively, and protrusions 610A, 610B of apparatus 600). In some embodiments, a neural lace apparatus can be manufactured using a first approach that is also referred to herein as the “Chiplet approach.” In this first approach, the neural lace apparatus can be manufactured such that one or more protrusions are made not of silicon but of polyimide, silicon carbide, and/or the like, with electronics affixed/mounted at junctions and/or beam corners of the polyimide sheet/mesh. For example, FIG. 12 shows a schematic representation of a fabrication process, following the first approach, to produce a neural lace apparatus with protrusions (also referred to herein as needles or cantilevered protrusions) made of polyimide, according to some implementations. The protrusions can be made using any suitable material including polyimide, silicon carbide, etc. The protrusions can be made to have any suitable dimension. For example, the protrusions can have a length from 0.1 mm to 2.0 mm, including any length therebetween. Alternatively or in addition, the protrusions can have a width from 20 microns to 1.0 mm, including any width therebetween.

FIG. 12 shows the first fabrication process 1200 for producing a neural lace apparatus using a silicon-based fabrication process. The process 1200 includes, at 1201, using a complementary metal oxide semiconductor (CMOS) wafer with aluminum (Al) pads (“D”) and a non-planarised passivation layer (“B”). In some instances, the CMOS wafer may be fabricated by doping a silicon substrate wafer to produce active wells (e.g., transistor sources, channels, and/or drains) that are subsequently selectively subjected to passivation. Alternatively, the CMOS wafer may be procured, such that the active electronics or the active CMOS elements are already incorporated in the CMOS wafer. The back end of the line (BEOL) passivation layer is configured to provide a layer of dielectric over the active electronics to dissipate surface charges and prevent such charges (e.g., static charges) from impacting the exposed electrical components (e.g., metal contact pads, amplifiers, and other electronics in the wafer). The passivation layer can comprise any suitable material (e.g., silicon nitride (SiN), silicon dioxide (SiO₂), etc.).

At 1202, the process 1000 includes transfer bonding or otherwise applying (e.g., via plasma deposition, chemical vapor deposition, etc.) a silicon dioxide (SiO₂) layer onto the CMOS layer that has undergone passivation. For example, the SiO₂ layer may be transfer bonded from another wafer on which the oxide layer has been thermally grown. Stated another way, the oxide layer may first be thermally grown on a first wafer (i.e., a thermal SiO₂ layer), and the thermal SiO₂ layer may then be transfer bonded onto the CMOS layer that has undergone passivation.

At 1203, the passivation layer is etched to expose the metal (e.g., Al) contact pads. At 1204, the process includes applying a layer of polyimide and etching out (e.g., via dry etching and/or wet chemical etching) the portions with underlying metal (e.g., Al pads) to again expose the metal pads. The polyimide layer can be etched from the portions overlying metal contact pads (and/or other contacts points defined to be open, for example, one or more electrical access sites that may be situated on the neural lace apparatus, including electrical access sites that may be situated on protrusions, etc.) via dry etching (e.g., plasma or chemical vapor etching).

At 1205, the process includes coating the wafer with one or more metals (e.g., titanium (Ti), platinum (Pt), a Ti/Pt bilayer, etc.) on the top surface. The metal coating layer can be configured (e.g., sufficiently thick and/or patterned) to provide electrical contact with/between the metal contact pads incorporated in the wafer. At 1206, the process 1200 includes applying a second layer of polyimide, optionally followed by dry etching the second layer of polyimide. The dry etching of the second layer of polyimide can be performed such that the metal contact pads and the metal coating layer are covered, however one or more metal electrical contact sites may be exposed (e.g., as a consequence of the dry etching) that are in electrical connectivity with the metal coat layer and the metal contact pads via the metal coat layer. These one or more metal electrical contact sites can serve as electrical access sites situated on a neural lace apparatus, for example situated on a protrusion of the neural lace apparatus, as shown at 1206 in FIG. 12 (see V-shaped inclusion). Also, at step 1206, the process 1200 includes grinding the silicon layer from below in portions that are not adjacent to the metal contact pads and that are predefined to form protrusions.

At 1207, after grinding away the silicon at 1206, protrusions remain. The protrusions (also referred to herein as “needles”) are the overhanging portions that include portions of the first polyimide layer and the second polyimide layer sandwiching apportions of the metal coating layer that is in electrical connectivity with one or more electrical access sites with exposed contact sites, as shown in FIG. 12. Each overhanging portion is configured to be flexible, bendable. or otherwise capable of being suitably manipulated (e.g., by providing an electrical voltage), such that each protrusion can be positioned in one or more desired poses, orientations, directions, etc., including directions that are not co-planar with (or are angled with respect to) the remaining portion of the wafer including the metal contact pads and/or active electronics.

The fabrication method of FIG. 12 can provide advantages in the form of ease of providing for protrusions with range extenders or protrusions with a length that is relatively long compared to the base portion (e.g., a protrusion that is longer than the diametrical distance between opposite vertices of a honeycomb structure. In some implementations, the wafer can include incomplete honeycomb structures with portions surrounding each honeycomb provided for generating protrusions with range extenders. Said in another way, in some embodiments, the protrusions can be substantially longer (e.g., protrusions with a first depth and a second depth greater than the first depth as described with reference to protrusions 610A with the first depth and protrusions 610B with the second depth in apparatus 600 in FIG. 6). In some embodiments, the protrusions can be configured to be patterned, during fabrication, such that they are displaced from the base structure of the neural apparatus with a bridging portion of polyimide, the bridging portion providing the desired length to form, upon release, the protrusions with the second depth. In some embodiments, a second set of the protrusions can be substantially longer than a first set of protrusions and so on. For example, protrusions with a first depth and a second depth greater than the first depth as described with reference to protrusions 610A with the first depth and protrusions 610B with the second depth in apparatus 600 in FIG. 6). In some embodiments, the protrusions can be configured to be patterned displaced from the base structure of the neural apparatus with a bridging portion of polyimide, the bridging portion providing the desired length to form, upon release, the protrusions with the second depth.

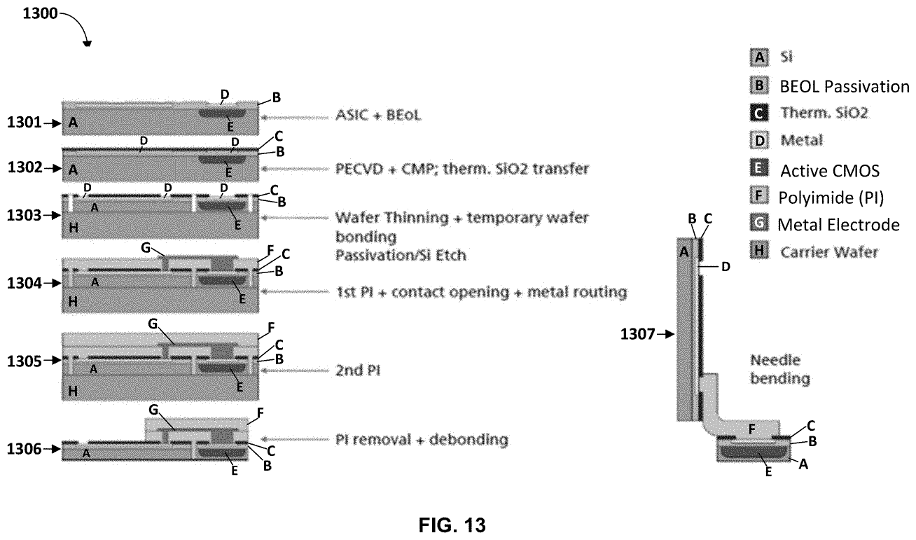

In some embodiments, a neural lace apparatus can be manufactured using a second approach where one or more protrusions can be made of silicon instead of polyimide. In some such embodiments, the protrusions made of silicon can include electronics in the structure of the protrusion. FIG. 13 shows a schematic representation of a second fabrication process for producing a neural lace apparatus with protrusions made of silicon, according to some implementations. The process 1300 includes, at 1301, using a complementary metal-oxide-semiconductor (CMOS) wafer including metal contact pads and ASIC elements. The CMOS wafer can include a passivation layer (“B”) that protects active components of the CMOS wafer from surface charges, as described herein.

At 1302, the process 1300 includes subjecting the wafer to plasma enhanced chemical vapor deposition (PECVD) and chemical mechanical polishing (CMP), followed by transfer bonding an SiOₑ layer that has been thermally grown on a second wafer. At 1303, the process 1300 includes thinning one side of the wafer and temporarily bonding the thinned side of the wafer to a temporary carrier (e.g., also a wafer, such as a silicon wafer) using any suitable bonding method. Also, at 1303, the process 1300 includes a passivation step followed by etching of the silicon to form “via” holes or trenches in the silicon layer at strategic/predefined locations and having predefined shapes, so as to form protrusions by bending or positioning portions of the wafer (post-fabrication) in non-planar directions. The vias or trenches can be of any suitable thickness and/or shape. For example, in some instances, the trenches can be defined to be approximately 50 micrometers (50 μm) thick and as deep as the full thickness of the remaining silicon layer.

At 1304, the process includes applying a first layer of polyimide such that the surface is coated, and the trenches are filled with polyimide. The first layer of polyimide can be applied using any suitable technique, including spin coating and/or chemical vapor deposition. The process then includes etching the polyimide at locations with underlying metal contact pads to provide access to the metal contact pads. Following the etching of the polyimide, the process at 1304 includes applying a metal routing layer (e.g., electrical “traces” or interconnect) that is electrically connected to the metal contact pads that are now exposed.

At 1305, the process 1300 includes applying a second layer of polyimide such that the metal routing layer is covered. At 1306, the process 1300 includes removing the first layer of polyimide and the second layer of polyimide at selected portions of the wafer, the selected portions predefined to form protrusions. The process then includes, at 1306, debonding the wafer from the temporary wafer. The debonded wafer can then be used to manipulate portions predefined to form protrusions and the remaining base portions of the neural apparatus (i.e., portions that are configured to be planar). Although shown as including polyimide within the via in the silicon layer at 1306, in other implementations, the polyimide may be selectively removed (e.g., via dry etching) from the via in the silicon layer. As shown on the right side of FIG. 13, the only connecting bridge between the base portions of a neural lace apparatus and the portions defined to form protrusions is a polyimide bridge (the depicted polyimide “elbow”) that is configured to be flexible and can be suitably bent at 1307 to form the protrusion as shown.

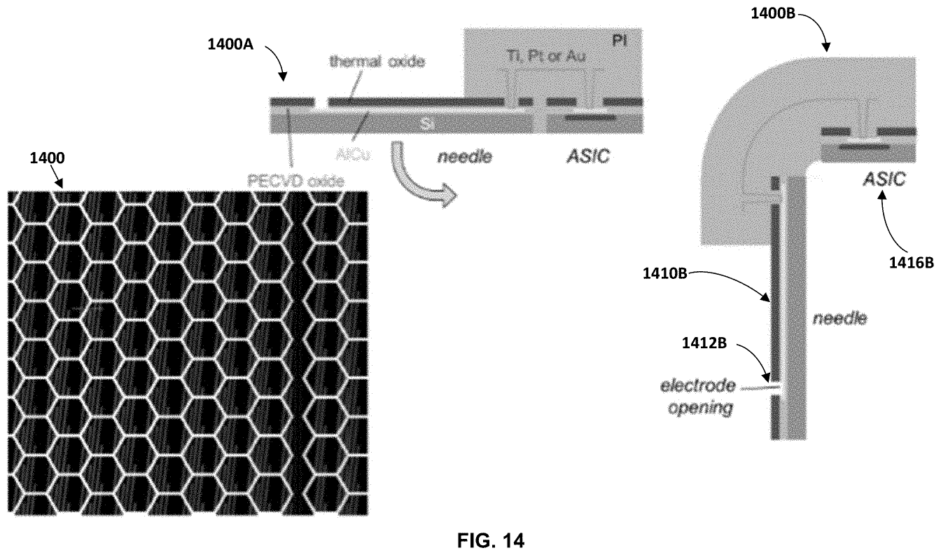

FIG. 14 shows two example configurations of protrusions of a neural lace apparatus, according to some implementations. The protrusions of FIG. 14 can be fabricated using any method described herein, for example with reference to FIG. 13. As shown in FIG. 14, the wafer 1400 includes a set of neural lace apparatuses manufactured in honeycomb pattern, with the vertices including active electronics and the protrusions configured to be pointing away from the vertices. The electronics (e.g., amplifiers, etc.) can be configured to be placed at nodal points or vertices of the honey comb structures. Some advantages of this approach can include the ability to perform relatively less complex and more efficient silicon processing, and the ability to test the wafer for useful dies with functioning components before transferring only the dies that are found useful. This approach, however, may also involve precision placement of components.

The wafer 1400 is shown in FIG. 14, for illustration purposes, as having regularly repeating honeycomb pattern, however in other embodiments the wafer can be patterned in any suitable manner to incorporate any suitable component (e.g., electronics, protrusions, etc.) of any suitable dimension.

A first protrusion configuration is shown at 1400A, in which controlling electronics (ASIC 1416A) routed to an internal metal conductive layer (e.g., comprising titanium, platinum, or gold (Ti, Pt, or Au)). The routing layer is insulated by the polyimide layer (PI). The protrusion configuration 1400A includes a thermal oxide layer (SiO₂). Upon release (and suitable manipulation) the apparatus 1400A can be transformed to the second configuration 1400B with the protrusion 1410B bent at a predefined angle away from the base portion of the neural lace apparatus. The protrusion 1410B can include one or more electrical access sites 1412B and the base portion can include active electronics 1416B.

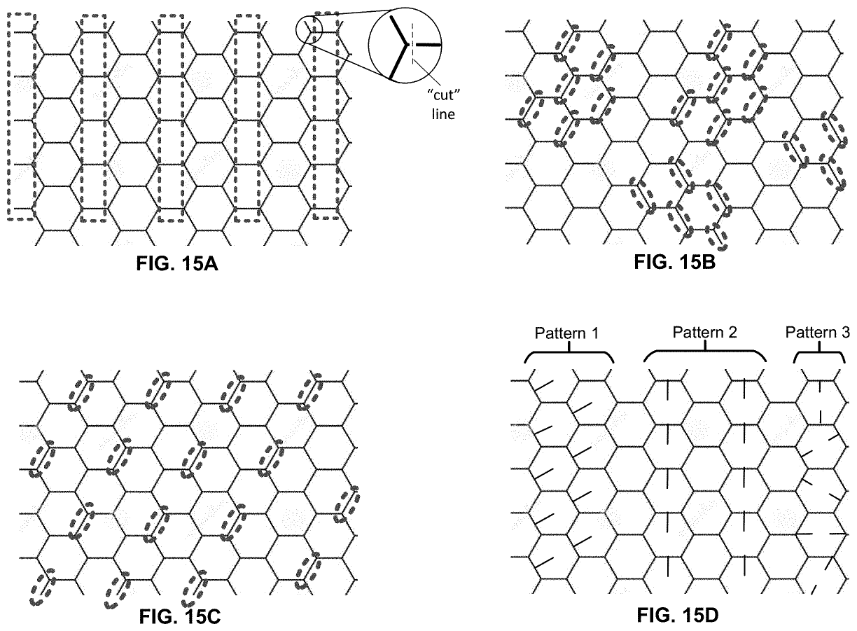

FIGS. 15A-15D show example arrangements of protrusions within a given neural lace apparatus, according to some embodiments. Protrusions are indicated by dashed circling in each of FIGS. 15A-15D. As shown in the inset of FIG. 15A, the protrusions may be “released” by way of “cut lines” that are engineered into the manufacturing process (e.g., via the lithography and/or etching steps). FIG. 15A shows protrusions arranged in regular linear arrays and formed from portions of the honeycomb structure itself. FIG. 15B shows multiple groupings of protrusions with differing orientations, also formed from portions of the honeycomb structure itself. FIG. 15A shows protrusions arranged in a diagonal array and formed from portions of the honeycomb structure itself. FIG. 15D shows three different protrusion patterns, formed from appendages to the honeycomb structure. Patterns 1 and 2 are arranged in regular/repeating linear arrays, while Pattern 3 exhibits protrusions having a variety of orientations, including appendages to the honeycomb structure that extend from linear members of the honeycomb structure and including appendages to the honeycomb structure that extend from vertices of the honeycomb structure.

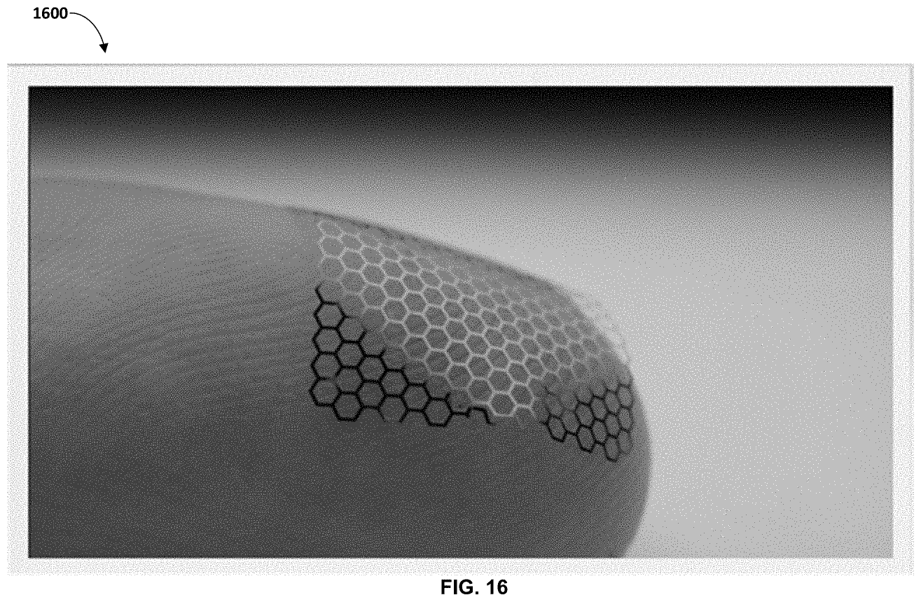

FIG. 16 is a photographic image of a neural lace apparatus 1500, according to an embodiment. The apparatus 1500 can be substantially similar in structure and/or function to the apparatus 100, 200, 300, 600, 700, and 800, described herein. The image depicts the dimensional features and the flexibility and delicate (yet robust for handling) properties of the apparatus 1500.

In some embodiments, a method for manufacturing neural lace apparatuses includes applying a first polyimide layer to a first planar surface of a silicon-based substrate, the silicon-based substrate including active components, passivation, and metal contacts, to form a first device structure. The method also includes etching one or more portions of the first polyimide layer to define openings therein. Each of the one or more portions of the first polyimide layer can overlie (e.g., be positioned directly over or overlapping with) at least one metal contact from the metal contacts, to form a second device structure. The method also includes depositing a metal layer to a surface of the second device structure, to define electrical interconnections among the metal contacts, thereby forming a third device structure. The method also includes applying a second polyimide layer to a surface of the third device structure, to form a fourth device structure. The method also includes performing a release process to produce a final device structure that includes a plurality of apparatuses (e.g., neural lace apparatuses), each apparatus from the plurality of apparatuses including a lace pattern and a cantilevered protrusion.

In some implementations, the silicon-based substrate includes at least one of a complementary metal-oxide-semiconductor (CMOS) substrate or an application-specific integrated circuit (ASIC).

In some implementations, the method also includes applying the passivation to the silicon-based substrate, prior to applying the first polyimide layer, by at least one of a thermal silicon dioxide transfer process or plasma-enhanced chemical vapor deposition.

In some implementations, the method also includes bonding the silicon-based substrate to a carrier prior to applying the first polyimide layer, and de-bonding the carrier from the silicon-based substrate after applying the second polyimide layer.

In some implementations, the method also includes thinning the silicon-based substrate from a second planar surface of the silicon-based substrate, the second planar surface of the silicon-based substrate opposite the first planar surface of the silicon-based substrate, prior to bonding the silicon-based substrate to the carrier.

In some implementations, the de-bonding includes dissolution of a wax bonding layer between the carrier and the silicon-based substrate.

In some implementations, the release process includes at least one of dry etching or wet etching silicon from a second planar surface of the silicon-based substrate, the second planar surface of the silicon-based substrate opposite the first planar surface of the silicon-based substrate.

In some implementations, the release process includes one of mechanically grinding or polishing silicon from a second planar surface of the silicon-based substrate, the second planar surface of the silicon-based substrate opposite the first planar surface of the silicon-based substrate.

In some implementations, the release process includes at least one of dry etching or wet etching portions of the first polyimide layer and the second polyimide layer.

In some implementations, the method also includes singulating each apparatus from the plurality of apparatuses from the final device structure.

In some implementations, the lace pattern includes a honeycomb structure, or any other structure shown and described herein.

While various embodiments have been described above, it should be understood that they have been presented by way of example only, and not limitation. Where methods and/or schematics described above indicate certain events and/or flow patterns occurring in certain order, the ordering of certain events and/or flow patterns can be modified. While the embodiments have been particularly shown and described, it will be understood that various changes in form and details can be made.

Although various embodiments have been described as having particular features and/or combinations of components, other embodiments are possible having a combination of any features and/or components from any of embodiments as discussed above.

In this disclosure, references to items in the singular should be understood to include items in the plural, and vice versa, unless explicitly stated otherwise or clear from the context. Grammatical conjunctions are intended to express any and all disjunctive and conjunctive combinations of conjoined clauses, sentences, words, and the like, unless otherwise stated or clear from the context. Thus, the term “or” should generally be understood to mean “and/or” and so forth. The use of any and all examples, or exemplary language (“e.g.,” “such as,” “including,” or the like) provided herein, is intended merely to better illuminate the embodiments and does not pose a limitation on the scope of the embodiments or the claims.

1. An apparatus, comprising:
a flexible electrically conducting layer disposed between a flexible first electrically insulating layer and a flexible second electrically insulating layer,
at least one of the first electrically insulating layer, the electrically conducting layer, or the second electrically insulating layer including a plurality of recesses defined therein, the plurality of recesses having a predefined pattern, and
a portion of the electrically conducting layer being exposed via an opening in at least one of the first electrically insulating layer or the second electrically insulating layer.

2. The apparatus of claim 1, wherein at least one recess from the plurality of recesses is partially defined in each of the first electrically insulating layer, the electrically conducting layer, and the second electrically insulating layer, such that the at least one recess extends through each of the first electrically insulating layer, the electrically conducting layer, and the second electrically insulating layer.

3. The apparatus of claim 1, wherein the predefined pattern includes one of a mesh or a lace geometry.

4. The apparatus of claim 1, wherein each recess from of the plurality of recesses has one of a square shape, a rectangular shape, a circular shape, an oval shape, or a polygonal shape.

5. The apparatus of claim 1, wherein the pattern includes a first pattern and a second pattern different from the first pattern.

6. The apparatus of claim 1, further comprising a protrusion including a portion of the electrically conducting layer and a portion of at least one of the first electrically insulating layer or the second electrically insulating layer, the protrusion extending along a direction that defines an angle of between 5° and 180° with respect to a plane defined by an adjacent portion of the apparatus.

7. The apparatus of claim 6, wherein the protrusion is tapered along a longitudinal axis thereof.

8. The apparatus of claim 6, wherein the protrusion includes a first longitudinal portion having a uniform thickness and a second longitudinal portion having a tapered thickness.

9. The apparatus of claim 1, wherein the protrusion has a substantially uniform thickness.

10. The apparatus of claim 6, wherein the opening is a first opening, the portion of the at least one of the first electrically insulating layer or the second electrically insulating layer including a second opening such that the underlying electrically conducting layer is exposed.

11. The apparatus of claim 6, wherein the protrusion is flexible.

12. The apparatus of claim 1, further comprising a plurality of protrusions, each protrusion from the plurality of protrusions including a portion of the electrically conducting layer and a portion of one of the first electrically insulating layer or the second electrically insulating layer

13. The apparatus of claim 1, wherein at least one of the first electrically insulating layer or the second electrically insulating layer comprises one of: polyimide, silicon carbide, SU-8, a liquid crystal polymer (LCP), Parylene-C, a ceramic, silicon dioxide, or any combination thereof.

14. The apparatus of claim 1, wherein the electrically conducting layer comprises one of: gold, platinum, iridium, iridium oxide, titanium nitride, poly(3,4-ethylenedioxythiophene (PEDOT), carbon nano-tubes (CNT), or a combination thereof.

15. The apparatus of claim 1, wherein the apparatus is configured to conform to a non-planar surface.

16. The apparatus of claim 1, further comprising at least one of: an integrated circuit, a controller, a multiplexer, a de-multiplexer, a buffer, an analog-to-digital converter, a digital-to-analog converter, a microelectronic coil, an ultrasonic emitter, an ultrasonic receiver, or an optoelectronic component.

17. A method, comprising:
providing an apparatus in a first, folded configuration, the apparatus including a flexible substrate having an electrically conductive layer disposed between a flexible first electrically insulating layer and a flexible second electrically insulating layer, at least one of the first electrically insulating layer, the electrically conducting layer, or the second electrically insulating layer including a plurality of recesses defined therein, the plurality of recesses having a predefined pattern; and
introducing the apparatus into a biological environment of a patient, thereby causing the apparatus to transition to a second, unfolded configuration, such that the apparatus substantially conforms to a non-planar surface within the biological environment of the patient.

18. The method of claim 17, wherein the introducing the apparatus into the biological environment of the patient is via a craniotomy.

19. The method of claim 17, wherein the introducing the apparatus into the biological environment of the patient includes passing the apparatus through a burr hole.

20. The method of claim 17, wherein the non-planar surface is a surface of the brain of the patient.

21-31. (canceled)
